(19) United States
(12) Patent Application Publication
(10) Pub. No.: US 2010/0215724 A1
(43) Pub. Date: Aug. 26, 2010

(54) Microcapsule Nanotube Devices for Targeted Delivery of Therapeutic Molecules

(75) Inventors: Satya Prakash, Brossard (CA); Hongmei Chen, Montreal (CA); Pavan Raja, Troy, NY (US); Omkaram Nalamasu, Niskayuna, NY (US); Pulickel M. Ajayan, Clifton Park, NY (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: McGill University

(21) Appl. No.: 12/085,017

(22) PCT Filed: Nov. 22, 2006
(86) PCT No.: PCT/US2006/045412

§ 371 Date: May 14, 2008

Related U.S. Application Data
(60) Provisional application No. 60/739,593, filed on Nov. 22, 2005.

(51) Int. Cl. A61K 9/127; A61K 9/10; A61K 39/395; A61K 38/02; A61K 31/7088; A61P 35/00

(52) U.S. Cl. 424/450; 424/484; 424/485; 424/486; 424/487; 424/488; 424/175.1; 514/2; 514/44 R; 977/742; 977/750; 977/752

(57) ABSTRACT

A nanotube device comprises a gel matrix that includes microcapsules and functionalized nanotubes, or other functionalized nanostructures incorporated into said gel matrix. Pharmaceutical compositions and methods of treatment comprising same. The pharmaceutical compositions of the present invention enable the specific and targeted delivery of therapeutic agents such as DNA molecules, peptides, including antibodies, drug molecules (e.g. small organic molecules), while offering sufficient resistance towards mucus layer of the intestine and high concentrations of enzymes and other molecules found in the blood stream and the GI tract.

### RELATED APPLICATIONS

This application claims the benefit of U.S. Provisional Application No. 60/739,593, filed Nov. 22, 2005. The entire teachings of the above application(s) are incorporated herein by reference.

### BACKGROUND OF THE INVENTION

Target-specific drug delivery systems are attractive therapeutical paradigms. These systems, however, face significant obstacles. For example, gene therapy has attracted wide attention as a method to treat various diseases. The present in vivo gene therapy systems suffers from hurdles such as lack of target specificity, problems of survivability of the DNA prior to reaching the target site, transformation efficacy of the DNA once it reaches the target site, etc. This is even more challenging in the case of oral delivery of DNA, due to the difficult gastrointestinal (GI) environment, for GI specific gene therapy applications.

To overcome these challenges, several methods have been proposed including encapsulation of DNA with microcapsules for targeted delivery. The microcapsules protect the encapsulated DNA from GI environment till they reach the target site where, upon degradation, they would release DNA molecules for gene therapy. Although the principle of using microcapsules is appealing, important concerns such as microcapsule stability, leakage of the encapsulated DNA before the target site is reached, sufficient resistance towards mucus layer of the intestine, lack of target specificity, etc., still exist, precluding the use of these devices in gene therapy applications. Similarly, carbon nanotubes (CNTs) has been proposed to be useful in delivery of therapeutic molecules, however, there application in GI targeted deliveries has not been possible. This invention deals with novel microcapsule carbon nanotube oral delivery device and their use in targeted delivery of therapeutic molecules. This novel device has various biomedical applications.

### SUMMARY OF THE INVENTION

The present invention relates to novel microcapsule carbon nanotube-based devices for oral administration. In particular, the devices of the present invention are suitable for oral administration of DNA for gene therapy, drug delivery and other applications.

In one embodiment, the present invention a carbon nanotube (CNT) device, comprising a gel matrix that includes microcapsules and carbon nanotubes incorporated into said gel matrix.

In another embodiment, the present invention is a pharmaceutical composition, comprising one or more carbon nanotube (CNT) device, including a gel matrix that includes microcapsules; and carbon nanotubes incorporated into said gel matrix in a pharmaceutically acceptable carrier.

In another embodiment, the present invention is a method of treating a patient suffering from toxins, comprising administering to said patient an effective amount of a composition comprising carbon nanotube (CNT) devices. The devices include a gel matrix that includes microcapsules; and carbon nanotubes incorporated into said gel matrix, or pharmaceutically acceptable thereof.

In another embodiment, the present invention is a method for treating cancer in a patient, comprising administering to the patient an effective amount of a composition comprising carbon nanotube (CNT) devices. The devices include a gel matrix that includes microcapsules; and carbon nanotubes or pharmaceutically acceptable therapeutic materials thereof, incorporated into said gel matrix and carbon nanotubes.

In another embodiment, the present invention is a composition of matter, comprising a gel slab that includes microcapsules; and functionalized carbon nanotubes incorporated into said gel slab, wherein the functionalized carbon nanotubes are selected from single wall or multiple carbon nanotubes.

In another embodiment, the present invention is a composition of matter, comprising a gel matrix that includes microcapsules; and particles incorporated into said gel matrix, wherein the particles include carbon nanotubes chemically altered by oxidation.

The pharmaceutical compositions of the present invention enable the specific and targeted delivery of therapeutic agents such as DNA molecules, peptides, including antibodies, drug molecules (e.g. small organic molecules), while offering sufficient resistance towards mucus layer of the intestine and high concentrations of enzymes and other molecules found in the blood stream and the GI tract.

### BRIEF DESCRIPTION OF THE DRAWINGS

FIG. 1 is a schematic representation of a molecular design of proposed novel microcapsule carbon nanotube devices and their possible application in delivery of DNA molecules to the intestinal epithelial cells in gene therapy applications.

FIGS. 2(a) and 2(b) is a schematic representation of non-covalent functionalization of CNT.

FIG. 2(c) is a schematic representation of one embodiment of a covalent functionalization of CNT.

FIG. 3 illustrates the transformation that takes place during the preparation of CNT (see Example 11).

FIGS. 4(a) through 4(e) are optical photomicrographs of the surfaces of alginate and alginate-polylysine capsules incorporating carboxylated (acid-treated) single-walled carbon nanotubes on their surfaces or inside their cores, using a 10× objective.

FIGS. 5(a) through 5(f) are SEM images of the surfaces of alginate and alginate-polylysine capsules incorporating carboxylated (acid-treated) single-walled carbon nanotubes on their surfaces or inside their cores.

FIG. 6 is a plot representing Raman spectra of some of the capsule samples of the present invention.

FIG. 7 is a schematic diagram depicting the process used for the preparation of the capsules of the present invention.

FIG. 8 is a schematic representation of the proposed functions of the (a) encapsulated and (b) surface-attached nanotubes.

FIG. 9. is a schematic representation of using proposed novel microcapsule carbon nanotube device for targeted delivery of APC gene to treat colon cancer. The device is functionalized with APC gene and anti-galectin 3 antibodies which selectively target cancer cells only which are known to overexpress galectin 3.

FIG. 10 is a series of photographs illustrating expression of Green Fluorescent Protein in SW480 cancer cells transfected with pAcGFP1-C1 plasmid using cationic liposome and carbon nanotube system. The cells that have been successfully transfected with the plasmid express the fluorescent protein and appear bright green when visualized under a Confocal Fluorescence Microscope. (A) SW480 cells; (B) SW480 cells transfected using Lipofectamine 2000; (C) SW480 cell transfected using chitosan-functionalized SWNT; (D) SW480 cells transfected with SWNT gene delivery system.

FIG. 11 is a schematic representation of using proposed novel microcapsule carbon nanotube device for targeted delivery of drugs to treat breast cancer.

FIG. 12 is a graph that shows the initial concentrations of HSA-paclitaxel, insulin and vitamin B12 used for functionalization and the concentration of each which was finally bound to the carbon nanotubes.

FIG. 13 is a graph shows the efficiency of binding of human serum bound paclitaxel, Insulin and Vitamin B12 on single walled carbon nanotubes for oral delivery using the designed novel microcapsule carbon nanotube device.

### DETAILED DESCRIPTION OF THE INVENTION

The present invention is a carbon nanotube device comprising a gel matrix that includes microcapsules; and carbon nanotubes incorporated either on the microcapsule surfaces and/or inside microcapsule cores and pharmaceutical compositions comprising same. The carbon nanotubes can further be functionalized by covalently or non-covalently attaching to a pharmaceutically active ingredient, such as a small molecule drug (organic molecules having molecular weight less than about 1000 D), a peptide or a DNA molecule.

Without being limited to any particular theory, it is believed that the nanotube devices, the pharmaceutical compositions and methods employing the nanotube devices of the invention serve one or more of the following functions: (i) promotion of attachment between the capsules or gels and the targeted cells; (ii) immobilization of the drug either directly onto the nanotubes, or entrapped between them and the gel matrix, for delayed release and enhanced encapsulation stability; (iii) adsorption of body toxins such as urea, creatinine etc. which are secreted in excess into blood or urine in the case of kidney failure; (iv) adsorption of matrix metalloproteinases (MMPs) secreted by metastasizing cancerous cells in the GI tract thereby limiting the spread of cancers of the GI tract; (v) alteration of the mechanical properties and degradation kinetics of the capsule or gel matrix at the site of targeted delivery either in vitro or in vivo.

Other applications of the pharmaceutical compositions that comprise carbon nanotubes of the present invention include tissue engineering constructs, biosensors, and industrial biocatalysts that employ immobilized enzymes.

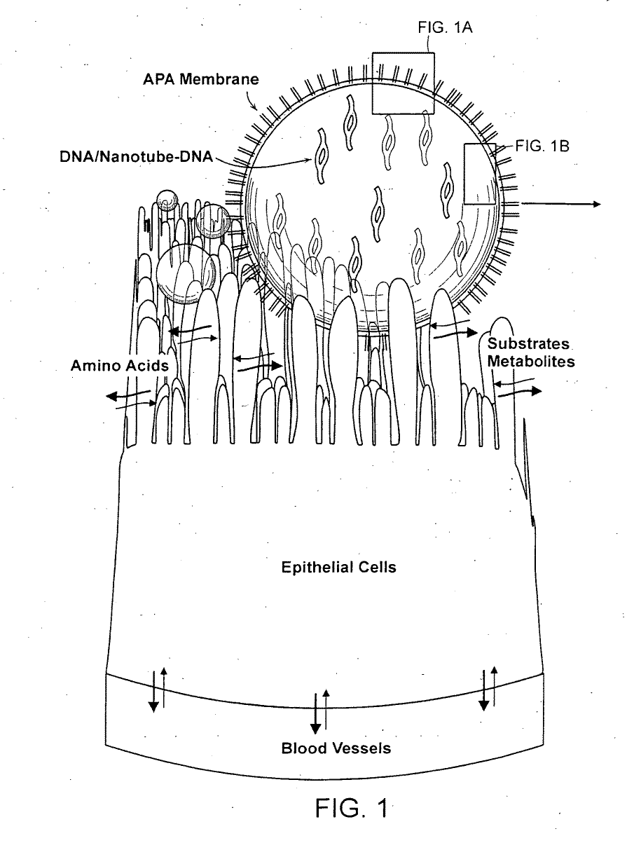

The molecular design of the microcapsules of the present invention is shown in FIG. 1. The material of the microcapsules is exemplified by alginate-poly-L-lysine-alginate (APA) microcapsules, described in details below. The APA membrane shown in FIG. 1 can be designed to possess a desired molecular permeability with appropriate molecular weight poly-lysine. For example, molecular permeability of 40,000 D will effectively protect the DNA form external enzymes such as DNAses, nucleases or any other biological molecules that have molecular weight larger than its permeability range.

### Carbon Nanotubes Suitable for Use in the Present Invention

A carbon nanotube is a thin graphene sheet rolled into a cylinder with both ends capped. These quasi-one-dimensional carbon whiskers are perfectly straight tubules with diameters of nanometer size, and properties close to that of ideal graphite. Single-walled nanotubes (SWNTs) with a cylindrical shell can be considered as the fundamental structural unit. Such structural units form the building blocks of multi-walled nanotubes (MWNTs), which contain multiple co-axial cylinders of ever increasing diameter about a common axis.

The hollow structure of nanotubes renders them lightweight as well as soft and flexible in axial bending and torsional tension modes. Carbon nanotubes are also very resistant to acidic media offering advantages for their use in use in GI delivery. Furthermore, carbon nanotubes can exhibit fully reversible large bending angles, an effect associated with the flexibility of the strongly bonded planar hexagonal network.

Furthermore, carbon nanotubes can be functionalized with proteins, lipids, and DNA molecules.

To functionalize CNTs with proteins by non-specific binding (NSB), they are suspended in an aqueous solution with SDS sonicated in 0.3% SDS for 1½ hours, diluted to 0.1% SDS, and centrifuged at 20,000 g for 30 min at 20° C. The gray supernatant is collected and incubated with 0.1 μg/μl of protein (e.g. streptavidin) overnight at room temperature, centrifuged again, and the supernatant is used. The binding of the protein to the CNT can be visualized using fluorescently-labeled proteins and fluorescence microscopy. A strong, homogeneously distributed signal along the nanotubes indicates efficient protein binding.

Alternately, irreversible adsorption of Tween 20 or triblock copolymer chains on nanotubes can prevent NSB, while at the same time enable the binding of specific proteins of interest that can be detected electronically without the need for labeling. To achieve this Tween 20 and 1,1-carbonyldiimidazole (CDI) are allowed to react in DMSO (dried under molecular sieve) at 40° C. for 2 hours with stirring. Ethyl ether is then added to effect precipitation, after which the precipitates are collected, redissolved in DMSO, and reprecipitated in ether. This process has to be repeated twice to ensure the removal of excess CDI and is followed by drying the intermediate in vacuo overnight. For conjugation to proteins, nanotube samples are first exposed to CDI-activated Tween (1 wt %, water) for 30 min, rinsed thoroughly with water to remove excess reagent, and then reacted with the protein of interest (e.g. biotin, streptavidin, staphylococcal proteins, antigens) in a sodium carbonate buffer (pH 9.5) for 24 hours at room temperature.

For functionalization of carbon nanotubes with lipids, COOH-modified CNTs are suspended in 20 mL of SOCl₂ and stirred at 65° C. for 24 hours. The solid is separated by filtration and distilled under decompression, followed by drying under vacuum at room temperature for 2 h, yielding COCl-modified SWNTs. In the final step, 1,2-bis(10,12-tricosadiynoyl)-sn-glycero-3-phosphoethanolamine (DCPE) is solublized in 10 mL of CHCl₃ and COCl-modified SWNTs with 1-[3-(dimethylamino)propyl]-3-ethylcarbodiimide hydrochloride (EDC) are added to the mixture, sonicating (20 kHz) for 30 min, and stirring at 50° C. for 48 h. The resulting solid is separated by vacuum filtration using a 0.22-μl Millipore polycarbonate membrane filter, washed with anhydrous alcohol several times, and dried overnight under vacuum. See, for example, Keren, K., Berman, R. S., Buchstab, E., Sivan, U. & Braun, E. DNA-templated carbon nanotube field-effect transistor. Science 302, 1380-1382 (2003); Zhang, S. Fabrication of novel biomaterials through molecular self-assembly. Nat. Biotechnol. 21, 1171-1178 (2003); Chen, R. J., Bangsaruntip, S., Drouvalakis, K. A., Kam, N. W. S., Shim, M., Li, Y., Kim, W., Utz, P. J. and Dai. H. Noncovalent functionalization of carbon nanotubes for highly specific electronic biosensors. PNAS 100 4984-4989 (2003); Baker, S. E., Lasseter, T. L., Smith, L. M. and Hamers R. J. Covalently-linked adducts of single walled nanotubes with biomolecules: Synthesis, hybridization and biologically-directed self-assembly. Mat. Res. Soc. Symp. Proc. 737 F4.6.1-F4.6.7 (2003); Wang, J., Kawde, A. N. & Musameh, M. Carbon-nanotube-modified glassy carbon electrodes for amplified label-free electrochemical detection of DNA hybridization. Analyst 128, 912-916 (2003); and Xin, H. & Woolley, A. T. DNA-templated nanotube localization. J. Am. Chem. Soc. 125, 8710-8711 (2003).

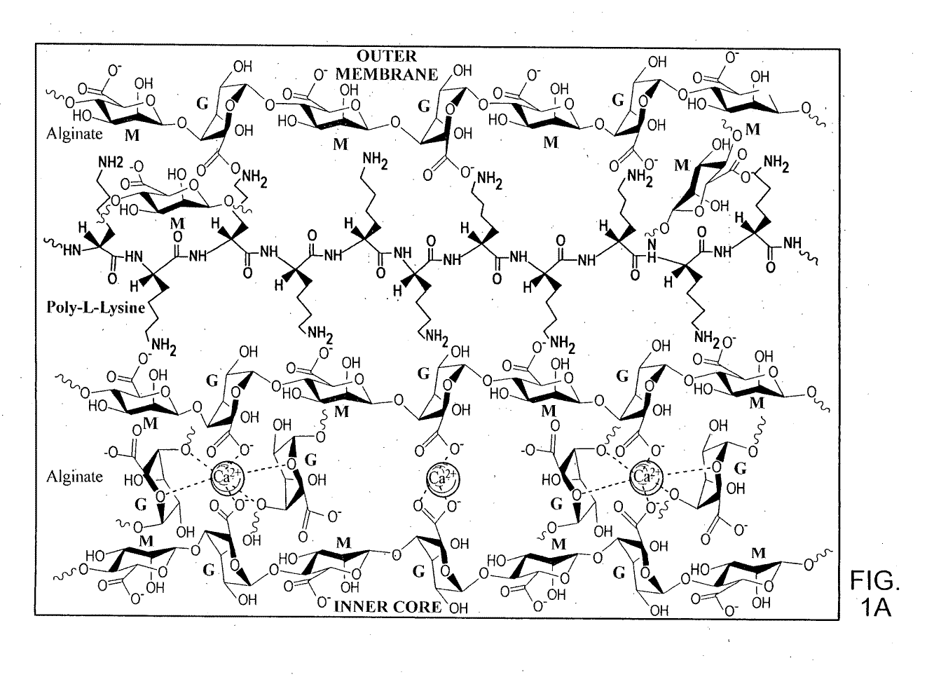

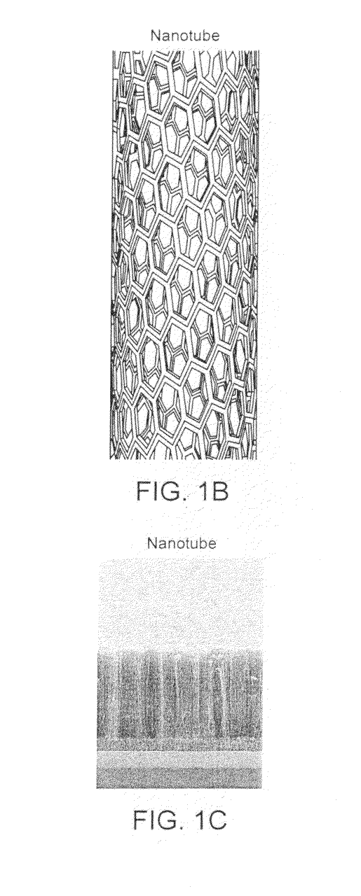

FIG. 2 illustrates the process of non-covalent functionalization of carbon nanotubes. Non-covalent functionalization of a CNT (either pristine or carboxylated) by a biomolecule (e.g. protein or DNA). The biomolecule is randomly adsorbed onto the CNT surface.

Ammonium-functionalized CNTs (f-CNTs) are able to bind with plasmid DNA and upon interaction with mammalian cells, these f-CNTs penetrate the cell membranes and are taken up into the cells efficiently; gene expression levels of up to 10 times higher were achieved than those obtained with plasmid DNA alone. For the generation of ammonium-functionalized CNTs, the carbon nanotubes are covalently modified by using a method based on the 1,3-dipolar cycloaddition of azomethine ylides. Amino acid and paraformaldehyde are added to a suspension of CNTs in dimethylformamide (DMF) and the mixture is heated at 130° C. for 96 h. After separation of the unreacted material by filtration, followed by evaporation of the solvent, the resulting residue is diluted with chloroform and washed with water. The combined organic phases are dried and the solvent is evaporated. Functionalized CNTs are isolated by precipitation with diethyl ether and the solid is subsequently washed several times with diethyl ether. Gaseous HCl is bubbled through a solution of these functionalized CNTs in dichloromethane (DCM), to remove the N-tert-butoxycarbonyl protecting group (Boc) at the chain-end. The corresponding CNT ammonium chloride salt precipitates during the acid treatment. After removal of the solvent, the brown solid is dissolved in methanol and precipitated with diethyl ether. See Pantarotto, D. et al. Functionalized carbon nanotubes for plasmid DNA gene delivery. Angew. Chem. Int. Ed Engl. 43, 5242-5246 (2004) and Kostarelos, K. Carbon nanotube-mediated delivery of peptides and genes to cells: translating nanobiotechnology to therapeutics. J. Drug Del. Sci. Tech., 15 (1), 41-47 (2005).

In some embodiments, the carbon nanotube devices of the invention can be functionalized by attaching a coupling agent moiety to the carbon structure. As used herein, a “coupling agent” is a chemical reagent that possesses at least two reactive moieties that allow to bond or link two or more molecules either covalently or non-covalently. Examples of the coupling agents include folate, albumin, and 1-ethyl-3-(3-dimethylaminopropyl)carbodiimide (EDAC). Using such coupling agents, a biologically active ingredient can be attached to a carbon nanotube. As used herein, the term “biologically active ingredient” means a compound, which, when administered to a subject in need of treatment, improves the prognosis of the subject, e.g., delays the onset of and/or reduces the severity of one or more of the subject's symptoms associated with a condition being treated. Examples of such biologically active ingredients include a small molecule, a peptide molecule and a DNA molecule. In one embodiment, the biologically active ingredient is a DNA molecule. In another embodiment, the biologically active ingredient is an antibody molecule. As used herein, the term “antibody molecule” includes an antibody (e.g. IgG, IgE, IgM, etc.) or a functional subunit of an antibody (e.g. Fc, Fab, F(ab)₂, CDRs, etc.).

In another embodiment, a biologically active ingredient may be included inside the carbon nanotube structure, in a clathrate-like manner. In this embodiment, a biologically active ingredient is a guest molecule within the carbon lettice of the nanotube.

In another embodiment, the present invention is a composition of matter, that comprises a gel matrix that includes microcapsules and particles incorporated into said gel matrix. The particles include carbon nanotubes chemically altered by oxidation.

Microcapsules Suitable for Use with the Present Invention

Microcapsules are useful as they protect the encapsulated materials from the external environment while at the same time permitting certain materials to pass into the microcapsules. They can also protect enzymes, cells, DNA etc. from adverse environments while allowing their delivery and use. The special feature of microencapsulation is, thus, the control provided by the choice of membrane. Microcapsules can be engineered for burst release, steady release, or to keep their contents in the body using appropriate membranes. The details of the process will be described below.

Numerous techniques for microencapsulation known in the art can be used with the present invention. In one embodiment, freeze-dried method of making membrane can be applied, using cellulose acetate phthalate (CAP) coated with beeswax. For encapsulation with beeswax, the cells are mixed with a solution of CAP; the resulting mixture is granulated with 30% w/w aqueous acacia mucilage and dried. Dried granules of uniform size (are further coated with molten beeswax. (See, e.g., Rao, A. V., Shiwnarain, N. & Maharaj, I. Survival of Microencapsulated Bifidobacterium-Pseudolongum in Simulated Gastric and Intestinal Juices. Canadian Institute of Food Science and Technology Journal-Journal de l Institut Canadien de Science et-Technologie Alimentaires 22, 345-349 (1989).)

In another embodiment, calcium alginate and κ-carrageenan-locust bean gum gel beads can be used for oral delivery. A solution of alginate and drug/cell suspension is mixed and stirred for 10 minutes to get a uniform mixture. The slurry is taken into a sterile syringe and added dropwise (or run through an encapsulator) into 0.2 M CaCl₂ solution and kept for curing at 4° C. for 1 hour. The cured beads are washed with sterile distilled water 3 to 4 times.

To prepare k-Carrageenan beads, aqueous dispersions of carrageenan are prepared in 15 ml distilled water. The drug is added while stirring. The beads are then prepared by dropping this dispersion from a disposable syringe into aqueous salt solutions stirred magnetically by using a magnetic stirrer. The beads formed are filtered under vacuum, washed with 100 ml distilled water and dried in a hot air oven at 37° C.] However, alginate beads are not acid resistant, and it has been reported that the beads undergo shrinkage and decreased mechanical strength. See, for example, Audet, P., Paquin, C. & Lacroix, C. Effect of Medium and Temperature of Storage on Viability of Lactic-Acid Bacteria Immobilized in Kappa-Carrageenan-Locust Bean Gum Gel Beads. Biotechnology Techniques 5, 307-312 (1991).

In another embodiment, gellan-xanthan beads can be used. Gellan and xanthan polymer powders are dispersed in deionized preheated water (80° C.) by gentle stirring and kept at 80° C. for 1 h. The temperature is then raised to 90° C. to achieve complete hydration of the polymers. The solutions are autoclaved for 15 min at 121° C. Cells from overnight (18-22 h) bifidobacterial cultures (20 ml) are harvested by centrifugation (5000 rpm, 15 min), washed and resuspended in 5 ml of sterile water. The bacterial suspension is mixed with 15 ml of gum solution so as to obtain a gel with the same initial cell concentration as the free cell culture and the desired gum concentration. Beads are manufactured by dropping polymer solution into 0.1 M CaCl₂ solution through a syringe needle (21 G) under gentle stirring. The formed beads are hardened in the CaCl₂ solution for 1 h and separated from the solution with a sterile stainless steel strainer. See for example, Norton, S., Lacroix, C. & Vuillemard, J. C. Effect of pH on the Morphology of Lactobacillus-Helveticus in Free-Cell Batch and Immobilized-Cell Continuous Fermentation. Food Biotechnology 7, 235-251 (1993).

In another embodiment, agarose microcapsules prepared by emulsification/internal gelationization can be used for oral delivery. Agarose microcapsules have been prepared by the emulsification of the hydrogel within a vegetable oil followed by its gelation due to the cooling of the system. Agarose capsules with different diameters, ˜70-1000 μm can also be prepared from aqueous agarose solution, using a droplets generator. The aqueous agarose solution is obtained by autoclaving the suspension of agarose powder in saline and extruded into a coflowing immiscible laminar flow of liquid paraffin containing 3 wt % lecithin from soybean, using a needle with an inner and outer diameter of 300 and 480 μm, respectively. The liquid paraffin, which is kept at 37° C., is allowed to flow in the same direction as the aqueous agarose solution. The diameter of the capsules can be controlled by changing the flow rates of the aqueous agarose solution and liquid paraffin. The resultant suspension is cooled to 4° C. in ice, to gel the agarose solution. After collection using mesh, the resultant capsules are washed several times with saline to remove the liquid paraffin from their surface.

In another embodiment, DNA was immobilized within alginate chitosan poly-L-lysine microcapsules to protect DNA hydrolysis from nucleases. DNA can been immobilized in alginate microspheres using the emulsification/internal or external gelation technique. A 2% (w/v) solution of sodium alginate containing 0.2% DNA is extruded through a syringe tip needle (0.001 in. internal diameter) apparatus into 50 mM calcium chloride solution. The technique is referred to as external gelation as the calcium source is external to the forming bead. To form internally gelled beads, 0.5% (w/v) suspended sonicated calcium carbonate powder is to be mixed with the alginate-DNA solution and extruded dropwise through a syringe needle into canola oil containing 0.2% (v/v) glacial acetic acid. The beads are reacted for at least 1 h prior to washing with 50 mM calcium chloride solution containing 1% (v/v) Tween-80 to remove excess oil. See, for example, Quong, D., Yeo, J. N. & Neufeld, R. J. Stability of chitosan and poly-L-lysine membranes coating DNA-alginate beads when exposed to hydrolytic enzymes. Journal of Microencapsulation 16, 73-82 (1999).

Other embodiments of microcapsules that can be used with the delivery system of the present invention include poly(lactide-co-glycolides), carrageenan, poly(D,L-lactic acid), alginate-poly-L-lysine, starch polyanhydrides, liposomes, polymethacrylates, polyamino acids such as poly-1-lysine, poly-1-ornithine, poly-1-arginine etc. See, for example, http://www.sigmaaldrich.com/Area_of_Interest/Biochemicals/PolyAmino_Acids.ht ml.

In other embodiments, enteric coating polymers cellulose acetate phthalate and polyvinyl acetate phthalate can be used. See, for example, Porter, S. C. Ridgway, K., “An evaluation of the properties of enteric coating polymers: measurement of glass transition temperature.” J Pharm Pharmacol. 1983 June; 35(6):341-4.

Several methods are used for the production of polymeric microcapsules, such as solvent evaporation, spray drying, homogenization, and sonication. The solvent evaporation method is preferable to other methods as it only requires ambient temperature conditions and mild emulsification techniques. The method is simple, with the microcapsules formed as an emulsion of the polymer/drug mixture. It usually involves a stirring process to shear the polymer/drug mixture into the continuous phase, generating product with significant polydispersity. Using pressurized microfluidic crossflow devices, monodisperse polymeric microspheres with tunable diameters can be produced.

Spray drying is a mechanical microencapsulation method in which an emulsion is prepared by dispersing the core material, usually an oil or immiscible with water, into a concentrated solution of wall material until the desired size of oil droplets are attained. The resultant emulsion is atomized into a spray of droplets by pumping the slurry through a rotating disc into the heated compartment of a spray drier to evaporate the aqueous portion, yielding dried capsules of variable shape containing scattered drops of core material. The capsules are collected through continuous discharge from the spray drying chamber.

In case of microencapsulation by phase inversion, drug is added to a polymer solution (1-4% w/v methylene chloride), which is then poured rapidly into an unstirred bath of non-solvent (petroleaum ether) at a solvent to non-solvent ratio of 1:100 causing nano and microspheres (0.1 to 5 μm) to form spontaneously.

In another method, drug loaded gelatin capsules are coated with various concentrations of sodium alginate and cross-linked, with appropriate concentrations of calcium chloride to protect the capsules from the harsh environment of the stomach. See Chen, H. M., Torchilin, V. & Langer, R. Polymerized liposomes as potential oral vaccine carriers: Stability and bioavailability. Journal of Controlled Release 42, 263-272 (1996); Chen, H. M. & Langer, R. Oral particulate delivery: status and future trends. Advanced Drug Delivery Reviews 34, 339-350 (1998); Chin, J., Turner, B., Barchia, I. & Mullbacher, A. Immune response to orally consumed antigens and probiotic bacteria. Immunology and Cell Biology 78, 55-66 (2000); Kreuter, J. Evaluation of Nanoparticles As Drug-Delivery Systems 0.3. Materials, Stability, Toxicity, Possibilities of Targeting, and Use. Pharmaceutica Acta Helvetiae 58, 242-250 (1983); Kreuter, J. Nanoparticle-Based Drug Delivery Systems. Journal of Controlled Release 16, 169-176 (1991); Narayani, R. & Rao, K. P. Preparation, Characterization and In-Vitro Stability of Hydrophilic Gelatin Microspheres Using A Gelatin-Methotrexate Conjugate. International Journal of Pharmaceutics 95, 85-91 (1993); Narayani, R. & Rao, K. P. Ph-Responsive Gelatin Microspheres for Oral Delivery of Anticancer Drug Methotrexate. Journal of Applied Polymer Science 58, 1761-1769 (1995); Narayani, R. & Rao, K. P. Polymer-Coated Gelatin Capsules As Oral Delivery Devices and Their Gastrointestinal-Tract Behavior in Humans. Journal of Biomaterials Science-Polymer Edition 7, 39-48 (1995); Narayani, R. & Rao, K. P. Gelatin microsphere cocktails of different sizes for the controlled release of anticancer drugs. International Journal of Pharmaceutics 143, 255-258 (1996); Gao, Y. T. & Wang, B. H. Safety comparison of insecticide microencapsulation and investigation of its mechanism. J. Microencapsul. 6, 527-533 (1989); Jones, D. H., Partidos, C. D., Steward, M. W. & Farrar, G. H. Oral delivery of poly(lactide-co-glycolide) encapsulated vaccines. Behring Inst. Mitt. 220-228 (1997); and Okazaki, K. et al. Intestinal drug delivery systems with biodegradable microspheres targeting mucosal immune-regulating cells for chronic inflammatory colitis. J. Gastroenterol. 37 Suppl 14, 44-52 (2002).

In one embodiment, the pharmaceutical composition of the present invention comprises calcium alginate beads with poly-L-lysine (PLL) forming alginate-poly-L-lysine-alginate (APA) microcapsules. Preparation of the alginate-poly-L-lysine-alginate (APA) microcapsule is carried out using non-toxic alginate derived from seaweed and a Inotech Encapsulator IE-20 (Inotech Biosystems International, Rockville, Md.). The alginate solution carrying drug, DNA or other material of choice is passed through the microencapsulator nozzle (different nozzle diameters can be used ranging from 80 to 400 μm) and dripped into a calcium chloride (CaCl₂) solution for gelation and form beads. The beads are allowed to gel for 5 minutes in a gently stirred sterile CaCl₂ solution, followed by a wash in physiological saline (PS). It is consequently coated with 0.1% sterile poly-L-lysine (PLL) followed by a wash with PS. A final coat of 0.1% sterile alginate follows; trailed by a final wash with PS. Such microcapsules are described in Ma, X. J., Vacek, I. & Sun, A. Generation of Alginate-Poly-L-Lysine-Alginate (Apa) Biomicroscopies—the Relationship Between the Membrane Strength and the Reaction Conditions. Artificial Cells Blood Substitutes and Immobilization Biotechnology 22, 43-69 (1994), referenced above, and Lanza, R. P. et al. Xenotransplantation of cells using biodegradable microcapsules. Transplantation 67, 1105-1111 (1999).

In the APA membrane microcapsule, alginate forms the core and matrix for the cell and PLL binds to the alginate core. Binding of PLL to alginate is the result of numerous long-chain alkyl-amino groups within PLL that extend from the polyamide backbone in a number of directions and interact with various alginate molecules, through electrostatic interactions. This resulting cross-linkage produces a stable complex membrane that reduces the porosity of the alginate membrane and forms a protective barrier.

In certain embodiments, APA microcapsules can be modified using a higher concentration of alginate cross-linked with barium instead of calcium, and the alginate can be fabricated as a gelled bead without solubilizing the core microcapsule. See, for example, Danielson, K. S. & Hunter, T. B. Barium capsules. AJR Am. J. Roentgenol. 144, 414 (1985); Peirone, M., Ross, C. J. D., Hortelano, G., Brash, J. L. & Chang, P. L. Encapsulation of various recombinant mammalian cell types in different alginate microcapsules. Journal of Biomedical Materials Research 42, 587-596 (1998); and Petruzzo, P. et al. Development of biocompatible barium alginate microcapsules. Transplant. Proc. 29, 2129-2130 (1997). APA membrane shows ghosting effects in the colon regions and breaks at pH levels above 6.5 making it suitable for colon targeted delivery.

### Pharmaceutical Compositions of the Present Invention and Methods for Administration Thereof

The present invention provides compositions for the treatment, prophylaxis, and amelioration of proliferative disorders, such as cancer. In a specific embodiment, a composition comprises one or more CNT devices of the invention, or a pharmaceutically acceptable salt, solvate, hydrate or prodrug thereof, and further includes one or more biologically active ingredient.

As used herein, the term “prodrug” means is itself biologically inactive but is converted in vivo to a biologically active compound.

In another embodiment, a composition of the invention comprises one or more prophylactic or therapeutic agents other than a device of the invention, or a pharmaceutically acceptable salt, solvate, hydrate, prodrug thereof. In another embodiment, a composition of the invention comprises one or more device of the invention, or a pharmaceutically acceptable salt, solvate, hydrate or prodrug thereof, and one or more other prophylactic or therapeutic agents. In another embodiment, the composition comprises a device of the invention, or a pharmaceutically acceptable salt, solvate, hydrate, or prodrug thereof, and a pharmaceutically acceptable carrier, diluent or excipient.

In a preferred embodiment, a composition of the invention is a pharmaceutical composition or a single unit dosage form. Pharmaceutical compositions and dosage forms of the invention comprise one or more active ingredients in relative amounts and formulated in such a way that a given pharmaceutical composition or dosage form can be used to treat or prevent proliferative disorders, such as cancer. Preferred pharmaceutical compositions and dosage forms comprise microencapsulated functionalized carbon microtubules of the present invention. The CNTs can be functionalized with an active compound including small molecule, DNA, antibodies or a pharmaceutically acceptable prodrug, salt, solvate, hydrate, or prodrug thereof, optionally in combination with one or more additional active agents.

A pharmaceutical composition of the invention is formulated to be compatible with its intended route of administration. Examples of routes of administration include, but are not limited to, parenteral, e.g., intravenous, intradermal, subcutaneous, oral (including inhalation), intranasal, transdermal (topical), transmucosal, and rectal administration. In a specific embodiment, the composition is formulated in accordance with routine procedures as a pharmaceutical composition adapted for intravenous, subcutaneous, intramuscular, oral, intranasal or topical administration to human beings. In a preferred embodiment, a pharmaceutical composition is formulated in accordance with routine procedures for subcutaneous administration to human beings.

Single unit dosage forms of the invention are suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form suitable for mucosal administration may contain a smaller amount of active ingredient(s) than an oral dosage form used to treat the same indication. This aspect of the invention will be readily apparent to those skilled in the art. See, e.g., Remington's Pharmaceutical Sciences (1990) 18th ed., Mack Publishing, Easton, Pa.

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms.

The suitability of a particular excipient may also depend on the specific active ingredients in the dosage form.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen (1995) Drug Stability: Principles & Practice, 2d. Ed., Marcel Dekker, NY, N.Y., 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizer” include, but are not limited to, antioxidants such as ascorbic acid, pH buffers, or salt buffers.

### Medical Applications of the Present Invention

Matrix metalloproteinases are enzymes associated with the invasive properties of tumour cells, owing to their ability to degrade all major protein components of the extracellular matrix (ECM) and basement membranes. They also play a role in early steps of tumour evolution, including stimulation of cell proliferation and modulation of angiogenesis. MMPs, such as MMP-11, suppress tumour cell apoptosis inhibiting cancer cell death. Overproduction of MMPs in tumour or stromal cells and cancer progression has been reported recently. Blocking the proteolytic effect of these enzymes has been proposed as a method of treating various cancers (e.g. breast, colon, lung).

Tissue inhibitors of metalloproteinases (TIMP) have been found to block the MMP activity in cancer and there are reports demonstrating their ability to inhibit tumour growth in transgenic mouse models. These are pseudopeptides containing a zinc-binding hydroxamate moiety which inhibits MMP activity by specifically interacting with the Zn²⁺ in the catalytic site.

The CNT devices of the present invention can be functionalized with (TIMPs) like Marimastat or Batimastat and used as an effective targeted delivery therapeutic to treat breast, colon and other cancers. SWNTs can be functionalized with a TIMP (e.g. Marimastat) and encapsulated in a suitable polymeric membrane for targeted delivery.

Alternatively, a gel matrix/slab of a hydrogel forming polymer (e.g. sodium alginate) can include with TIMP-functionalized carbon nanotubes (either in free form or encapsulated in polymeric membrane) and used for selective removal of MMPs from the extra-cellular matrix (ECM). (See KRUGER, A., FATA, J. E. and KHOKHA, R. (1997). Altered tumor growth and metastasis of a T-cell lymphoma in Timp-1 transgenic mice. Blood. 90: 1993-2000; MARTIN, D. C., SANCHEZ-SWEATMAN, O. H., HO, A. T., INDERDEO, D. S., TSAO, M. S, and KHOKHA, R. (1999). Transgenic TIMP-1 inhibits simian virus 40 T antigen-induced hepatocarcinogenesis by impairment of hepatocellular proliferation and tumor angiogenesis. Lab. Invest. 79: 225-34; BOULAY, A., MASSON, R., CHENARD, M. P., EL FAHIME, M., CASSARD, L., BELLOCQ, J. P., SAUTES-FRIDMAN, C., BASSET, P. and RIO, M. C. (2001). High cancer cell death in syngeneic tumors developed in host mice deficient for the stromelysin-3 matrix metalloproteinase. Cancer Res. 61: 2189-93.)

### Additional Uses of the Present Invention

In addition to the application described herein and, in greater details, in Exemplification, the microcapsules of the present invention can be used for immobilization of proteins and/or other biomolecules in the capsule/hydrogel cores or surfaces for sustained industrial biochemical reactions; for assays pertaining to the configuration or sorting of biomacromolecules immobilized in the capsules/hydrogels, with or without incorporating carbon nanotubes, under different physicochemical or electrical conditions; for tissue engineering applications based on hydrogels incorporating (or reinforced by) carbon nanotubes; and for cell toxicity assays based on intimate interactions between cells and nanotubes entrapped in hydrogels.

Self assembly and/or directed assembly of nanotubes or nanoparticles embedded in hydrogels, their characterization, and application as gaseous, chemical/biochemical or physical sensors. Model systems to study the mechanical properties of cross-linked polymeric systems with and without the incorporation of carbon nanotubes or other nanostructured materials as reinforcing agents.

### EXEMPLIFICATION

### Example 1

### Fabrication and Characterization of Carbon Nanotubes

Carbon nanotubes are prepared by any one of the following three methods.

(1) Arc-discharge (AD) method utilizes a graphite rod as carbon source and the reaction is triggered by arc discharge from high density current. This method can produce SWNTs and MWNTs. The main advantage of arc grown MWNTs is that they can be grown without any metal catalyst. This makes them especially attractive for in-vivo applications. The SWNTs usually contain up to 50% amorphous carbon and 5% metallic catalyst but they can be easily purified of metals by established processes.

(2) Chemical vapor deposition (CVD) method is considered a better choice to produce controllable nanotubes. There are several advantages for CVD process: (a) CVD process is more controllable, the reaction process can be controlled by adjusting temperature, pressure or chemical vapor precursor, thus the purity and morphology of carbon nanotubes can be optimized. (b) The catalysts such as DNA can be easily applied. They can be applied on substrates by dip coating, or mixed with chemical precursor directly. (c) CVD process is easily scaled up for mass production. Because CVD process provides more flexibility for the conversion of carbon nanotubes from chemical vapor precursor, this process can be scaled up for manufacturing by expanding the reactor size or by increasing the number of reactors. Nanotubes are preferably 0.8 to 1.2 nm in diameter, and have lengths ranging between 100 and 1000 nm.

(3) Laser ablation is another method for producing carbon nanotubes. This method can produce relatively higher purity nanotubes but it still suffers several problems such as size control and are difficult to scale up for mass production and is not used. The nanotubes are characterized using Raman spectroscopy, FTIR spectroscopy and UV-VIS spectrophotometry, Scanning Electron Microscopy (SEM) and Transmission Electron Microscopy (TEM). Presence of catalyst particles and trace elements in the nanotubes are analyzed using Energy Dispersive X-ray (EDX) feature of the SEM available in the McGill EM Center.

### Example 2

### Functionalization of Carbon Nanotube with DNA of Interest for Targeted Delivery

The above-prepared nanotubes are made hydrophilic by ultasonicating them in a sulfuric and nitric acid mixture (3:1) for a suitable period of time to functionalize nanotube sidewalls and tips with hydrophilic carboxylic acid and hydroxyl groups to form stable suspensions in water and to remove unwanted iron catalyst particles entrapped inside the nanotubes.

Once this is achieved, nanotubes are used for subsequent functionalization. Suitable functionalization techniques are described, for example, in Kostarelos, K. Carbon nanotube-mediated delivery of peptides and genes to cells: translating nanobiotechnology to therapeutics. J. Drug Del. Sci. Tech., 15 (1), 41-47 (2005).

Carbon nanotubes are first covalently modified by using a method based on the 1,3-dipolar cycloaddition of azomethine ylides to yield ammonium functionalized CNTS. An aqueous solution of these ammonium functionalized CNTs (720 μg mL⁻¹) is mixed with plasmid DNA (5 μg mL⁻¹) in a 6:1 (+/−) charge ratio to yield DNA functionalized CNTs. In the embodiments that employ the devices of the present invention for DNA delivery, carbon nanotubes are functionalized with plasmid DNA. Plasmid DNA is covalently (by the method of Williams, K. A., Veenhuizen, P. T., de la Torre, B. G., Eritja, R. & Dekker, C. Nanotechnology: carbon nanotubes with DNA recognition. Nature 420, 761 (2002)) or non-covalently (by the method of Pantarotto, D. et al. Functionalized carbon nanotubes for plasmid DNA gene delivery. Angew. Chem. Int. Ed Engl. 43, 5242-5246 (2004))) attached to the carbon nanotubes. Non-covalent scheme is preferred. The functionalized nanotubes is characterized using standard techniques such as TEM and AFM (nanotube morphology), FTIR, UV-VIS spectrophotometry and SDS-PAGE analysis.

### Example 3

### Methods for Making Microcapsule Carbon-Nanotube Devices

Functionalized nanotubes are suspended in polymer matrix (e.g. alginate). The viscous polymer-nanotube DNA suspension is then pressed through a 23-gauge needle using a syringe pump. Compressed air press through a 16-gauge needle is used to shear the droplets coming out of the tip of the 23-gauge needle. The droplets are allowed to gel for 15 minutes in a gently stirred ice-cold solution of solidifying chemicals, such as CaCl₂ (1.4%).

After gelation in the CaCl₂, beads are washed with HEPES (0.05%, pH 7.20) and coated with poly-L-lysine (0.1% for 10 min) and wash again in the HEPES (0.05%, pH 7.20). The resulting capsule is then be coated with poly-L-lysine (0.1%) for 10 minutes followed by alginate for 30 minutes. After another HEPES (0.05% in HEPES, pH 7.20) washing, the resultant capsule is finally coated for 10 min with alginate (0.1%). The microcapsules are then washed with appropriate chemicals to dissolve their inner core content. For this a 3.00% citrate bath (3.00% in 1:1 HEPES-buffer saline, pH 7.20) is be used. An automated Inotech Encapsulator is used to prepare large quantity of microcapsule.

To incorporate the nanotubes at the surface of the microcapsule for developing the microcapsule nanotube delivery device, either singlewalled or multiwalled carbon nanotubes can be used.

Nanotubes that are rendered water soluble by functionalizing them with simple organic moieties like carboxyl and hydroxyl groups on the side-walls as well as on the tip of the nanotube are used. This is achieved by ultrasonication of the nanotubes in a sulfuric and nitric acid mixture (3:1) for a few hours. Metal catalyst particles usually get dissolved away during this process. The acid treated and functionalized nanotubes are easily dissolved in water and acetone and incorporated in the APA membrane during the final coating of poly-1-lysine (in HEPES) on the microcapsule.

Nanotubes with carboxyl function groups are coupled to the amine group of poly-1-lysine to form amide bonds using carbodimide coupling agents. An example of such coupling is shown in FIG. 2(c), which illustrates covalent functionalization of carboxylated CNTs with a poly-1-lysine (PLL) molecule, using a carboxylate activator such as EDC. The PLL molecule is covalently attached to the CNT by forming an amide linkage between its amine group and the CNT's carboxylate group.

This gives further stability to the nanotube-APA composite membrane. The grown nanotubes SWNTs and MWNTs are 1-3 μm long but they can broken down to shorter length scales (100-500 nm) by ultrasonication in concentrated acid mixture of sulfuric and nitric acids. The length can be varied by altering the acid concentration as well as the sonication time. The nanotubes incorporated at the surface of the microcapsule can be further functionalized as described in Example 2.

### Example 4

### Evaluation of Microcapsule Nanotube Devices DNA Delivery Transfection Efficacy In-Vitro

Model cell lines such as intestinal epithelial cell system, IEC-6 cells, ATCC catalog no. CRL-1592, or colon cancer cell lines HCT-116(p532/2) and HCT-116(p531/1) (from ATTCC) and SAOS-2 cell lines (from National Institute of Aging (NIA) can be used as a target for transfection by the nanotube-DNA complexes in vitro.

To evaluate the transfection efficacy of the target cells with the microencapsulated nanotubes of the present invention functionalized to carry DNA, human colon cancer cell lines HCT-116(p532/2) and HCT-116(p531/1) (from ATTCC) and SAOS-2 cell lines (from NIA) are grown in McCoy's 5a and Dulbecco's modified Eagle's medium, respectively, supplemented with 10% fetal bovine serum (Life Technologies, Inc.) at 37° C. under a humidified atmosphere of 5% CO2.

After cells reached 70% confluence, fresh medium without fetal bovine serum is added to each dish and then further incubated for an additional 20 hours. Once the cell reaches to 60% confluence in 60-mm tissue culture dishes, microcapsule nanotube device containing approximately 1.5 mg/ml of APC plasmid and 14 mg/ml of the LipofectAMINE reagent is added as recommended by the manufacture. After 24 h of post-transfection, cells are starved in a serum-free medium for an additional 18 hours and treated with DNA alkylating agents for 50 h. After the treatment, cellular lysates are prepared for LacZ activity. Quantitative analysis of the data is done by electronic autoradiography using Instant Imager from Packard Instrument Company (Meriden, Conn., USA).

### Example 5

### Microcapsule Carbon Nanotube Devices Characterization

The microcapsule formulations are characterized for their physiological, biochemical, functional and other properties in-vitro.

### (1) Microcapsule Carbon Nanotube Devices Morphology Study

Microcapsule carbon nanotube devices morphology are determined using optical microscopy and scanning electron microscopy (SEM). The effect of processing variables of chemical concentrations, drying conditions, concentration of hardening agent and hardening time on the bead properties as well as the release characteristics of nanocapsules is studied. The size distribution, geometry, release mechanism, storage stability, and other parameters are also be evaluated. Further, a comparative characteristic of microcapsules by SEM and swelling dynamics under varied pH and other conditions found in GI tract are assessed.

### (2) Microcapsule Carbon Nanotube Device Molecular Permeability Studies

Molecular permeability and GI DNA's protection capacity of formulations of the present invention is determined using size exclusion chromatography (SEC).

### (3) Microcapsule Carbon Nanotube Devices DNA Leakage Study

Carbon nanotubes functionalized with plasmid DNA molecules are encapsulated in alginate capsule cores and subjected to different pH environments (pH 2-10) simulated at body temperature (37° C.) for different lengths of time (1, 4, 8, 12 and 24 hours) on a rotary shaker equipped with temperature control. The supernatant liquid surrounding the capsules is monitored by UV-VIS spectrophotometer and SDS-PAGE, to detect microcapsule carbon nanotube device DNA leakages.

### (4) Microcapsule Carbon Nanotube Device Bioadhesive Studies Using a Tensile Technique

To evaluate if microcapsule carbon nanotube devices actually adhere to the intestinal epithelial cells, the adhesive forces between microcapsule carbon nanotube device and segments of intestinal tissue are measured using a Cahn Dynamic Contact Angle Analyzer Model DCA-322. Although this equipment is designed for measuring contact angles and surface tensions using the Wilhelmy plate technique, it is also an extremely accurate microbalance and provides methods to evaluate comprehensive understanding of the complex interactions at liquid-liquid and liquid-solid surfaces. The DCA-322 system includes a microbalance stand assembly, a computer, and an Okidata Microline 320 dot matrix printer. The microbalance unit consists of stationary sample and tare loops and a moving stage powered by a stepper motor. The balance can be operated with samples weighing up to 3.0 g, and it has a sensitivity rated at 0.001 dynes. The stage speed can be adjusted from 20 to 264 μm/sec using a factory installed motor or from 2-24 μm/sec using the optional slow.

Adhesive forces are measured by attaching a polymer sample to one of the sample loops and placing an adhesive substrate, intestinal tissue, below it on the moving stage. For each adhesive measurement, 1.5 cm sections are cut from the excised intestine. These are then sliced lengthwise and spread flat, exposing the lumen side. The samples are then placed in a temperature-regulated chamber after clamping them at their edges, and covered with approximately 0.9 cm high level of phosphate buffer saline. Physiological conditions are maintained in the chamber through experiment period. The chamber is then be placed in the microbalance enclosure and a microcapsule carbon nanotube device mounted on a wire and hung from the sample loop of the microbalance is in contact with the tissue. The microcapsule nanotube device is left in contact with the tissue for 7 minutes with an applied force of approximately 0.25 mN and then it is pulled vertically away from the tissue sample and force for detachment is recorded.

### (5) Cell Viability Cytotoxicity and Immunotoxicity Studies

Formulation cytotoxicity activity are evaluated by using the MTT (3-(4,5-dimethylthiazol-2-yl)-2,5-diphenyl tetrazolium bromide) colorimetric assay and the cell viability by using the Trypan blue dye exclusion method, described in Savi, L. A. et al. Evaluation of anti-herpetic and antioxidant activities, and cytotoxic and genotoxic effects of synthetic alkyl-esters of gallic acid. Arzneimittelforschung. 55, 66-75 (2005) and Rodil, S. E., Olivares, R. & Arzate, H. In vitro cytotoxicity of amorphous carbon films. Biomed. Mater. Eng 15, 101-112 (2005). The cytotoxicity of carbon nanotubes can be evaluated using the Promega CellTiter 96® AQᵤₑₒᵤₛ Non-Radioactive Cell Proliferation Assay kit (MTS assay). This uses 3-(4,5-dimethylthiazol-2-yl)-5-(3-carboxymethoxyphenyl)-2-(4-sulfophenyl)-2H-tetrazolium, inner salt; MTS, a tetrazolium compound) and the electron coupling reagent, phenazine methosulfate (PMS). MTS is chemically reduced by cells into formazan, which is soluble in tissue culture medium. The dehydrogenase enzyme activity found in metabolically active cells is measured in terms of light absorbance values in this assay. Mammalian cells (e.g. SW 480 colorectal cancer cells, MDA-MB-231 breast cancer cells) are seeded onto a 96-well plate at the rate of 10⁴ cells per well for the evaluation of cytotoxicity by MTS assay. The cells are then exposed to the formulation and the MTS assay reagents over an extended time period. The measurement of the absorbance of the formazan was carried out using the 1420-040 Victor³ Multilabel Counter (Perkin Elmer) at 490 nm. Samples where collected from cell wells and measured at periodic intervals after exposure to the formulation. Measurements are done in triplicate.

Since the production of formazan is proportional to the number of living cells, the intensity of the produced color is an indicator of the viability of the cells. The measurement of the absorbance of formazan produced was carried out using the Victor Multilabel Plate Reader at 490 nm. Since the production of formazan is proportional to the number of living cells, the intensity of the produced color is an indicator of the viability of the cells.

Immunomodulatory effects of the formulations of the present invention is analyzed by detection of pro-inflammatory and down-regulatory cytokines in supernatants of thioglycollate-elicited peritoneal macrophages using enzyme-linked immunosorbent assay (ELISA). This microcapsule formulation is incubated with macrophages and increase production of interleukin (IL)-6, IL-12, and tumor necrosis factor (TNF)-alpha is evaluated. Any decrease in cytokine production or increase in IL-6 is attributed to evaluate immunoxicity of microcapsule formulation.

### Example 6

### Microcapsule Carbon Nanotube Device Stability Studies in Various GI Enzymatic Conditions

In-vitro studies are performed in the flask to evaluate the susceptibility of microcapsule carbon nanotube device towards various GI tract enzymatic conditions. Not only GI tract enzymes but also other enzyme that microcapsules are likely to be challenged with, are evaluated for microcapsule carbon nanotube device enzymatic stability. In particular enzymes such as DNAses, nucleases, deoxyribonucleases, pepsin, intestinal amylase, pancreatic trypsinogen, chymotrypsinogen, carboxypeptidase, amylase, and alkaline phosphatase are evaluated.

All the experiments are carried out using microcapsule carbon nanotube device containing DNA and empty microcapsule carbon nanotube device in a 250-ml flask at 30° C. and 100 rpm shaking.

### Example 7

### Microcapsule Carbon Nanotube Device GI Stability Studies in Computer Controlled GI Model

### (1) Preparation of Computer Controlled Dynamic In-Vitro GI Tract Model

The formulations of the present invention are tested in a simulated human microbial ecosystem (GI tract model) has been prepared as described by Prakash, S. & Chang, T. M. S. Preparation and In-Vitro Analysis of Microencapsulated Genetically-Engineered Escherichia-Coli Dh5 Cells for Urea and Ammonia Removal. Biotechnology and Bioengineering 46, 621-626 (1995), with some modification.

In brief, a sixth reactor vessel simulating the stomach is connected to the five reactors in the original model. Each of the six reactor vessels represents distinct parts of the human GI tract in the following order (reactors 1-6): the stomach, the duodenum, the small intestine, the ascending colon, the transverse colon and the descending colon. Each reactor vessel has eight ports: for input and output of the medium, sampling of liquid phase, gas, pH electrode, pH control (acid and base), and for flushing of head space. The pH of the reactor 2, 3, 4 and 5 is controlled between 6.5 and 7.0, 5.5 and 6.0, 6.0 and 6.4, 6.4 and 6.8 respectively using 0.5M NaOH, and 0.5M HCl. The GI model is fed three times a day with feed medium comprised of glucose 0.4 g/day, arabinogalactan 1 g/day, pectin 2 g/day, xylan 1 g/day, starch 3 g/day, yeast extract 3 g/day, peptone 1 g/day, mucin 4 g/day, and cysteine 0.5 g/day.

All experiments are carried out at room temperature. All physiological and biochemical parameters of the model, including transfer of content from one vessel to other, are computer controlled using a Lab View 61 software. This in vitro system closely mimicking the in-vivo conditions with regard to pH, bacteria, types of enzymes, enzymatic activity, volume, stirring, and possible food particles.

### (2) In-Vitro Experimental Procedure in Computer Controlled Dynamic GI Tract Model

Six different biochemical property reactor vessels are prepared and activated. A known quantity of microcapsule carbon nanotube device is added and allowed to react in vessel 1 for a fixed period of time and then is passed to reactor 2,3,4,5, and 6 after a certain time interval using peristaltic pump controlled by Lab-View 6i. Sample microcapsule carbon nanotube device is taken out at different intervals form each vessel (for up to 72 hrs, the maximum time a food can be retained in the human GI). At fixed time intervals various microcapsule carbon nanotube device properties are evaluated in each GI compartments to assess the over all fate of each type of device when given orally.

### Example 8

### In-Vivo Transit Time Studies Using X-Ray Imaging

Studies are performed to determine the microcapsule in-vivo transit time, incidences of prolonged transit and other effects. For this, special microcapsule nanotube device with radiopaque markers is prepared. A group of mice is fed with control (no marker) and test devices (loaded with marker). For each rat, 100 mg microcapsule nanotube device containing marker is suspended in 2 ml of H₂0 and they are force-fed using a gavage. Pure radiopaque markers suspension in distilled H₂0 is fed to the control group of mice. At given time intervals, the mice are X-rayed and the distribution of the microcapsule device in the GI is followed and evaluated for delays in their passage. In addition, after fixed hours after feeding, the mice are sacrificed and their intestines opened. Microcapsule nanotube device are then located in the intestine. The areas with spheres adhering to the tissue are washed with saline. The tissue is fixed in neutral 10% formaldehyde solution for 24 hr. After fixation, the tissue is exposed to increasing concentrations of alcohol solutions, starting from 50:50% water and ethanol, and ending with 100% ethanol. The dry samples is then coated with gold-palladium and analyzed under a SEM for morphological studies.

### Example 9

### Study Evaluating Nonspecific Uptake of Microcapsule Carbon Nanotube Devices

Cells normally internalize soluble ligands and small particles via endocytosis and large particles via actin-based phagocytosis. This is performed as in-vitro as well as in-vivo experiments. In in-vitro studies microcapsule nanotube devices containing fluorescently labeled polystyrene beads (2% w/v, 20-30 nm) are prepared and test uptake using ATCC mouse squamous cell carcinoma cell lines are used.

For in-vivo studies, nanotubes encapsulated in polystyrene beads (2% w/v, 20-30 nm) is administered by gavage to APC mice daily for 10 days at a dose of 1.25 mg/kg-1 and perform histological evidence of the uptake of these particles and their absorption across the GI tract and passage via the mesentery lymph supply and lymph nodes to the liver and spleen by analysis of tissues for the presence of polystyrene by HPLC gel permeation chromatography.

### Example 10

### Preparation of Carbon Nanotube Microcapsules

Low viscosity sodium alginate (Sigma Chemical Co., catalog no. A-2158), calcium chloride (Aldrich Chemical Co., catalog no. 22231-3) and poly-1-lysine (M_{w}=27400 D, M, w Mₙ=1.3, Sigma Aldrich Chemical Co., catalog no. P-7890) were used as obtained from the suppliers.

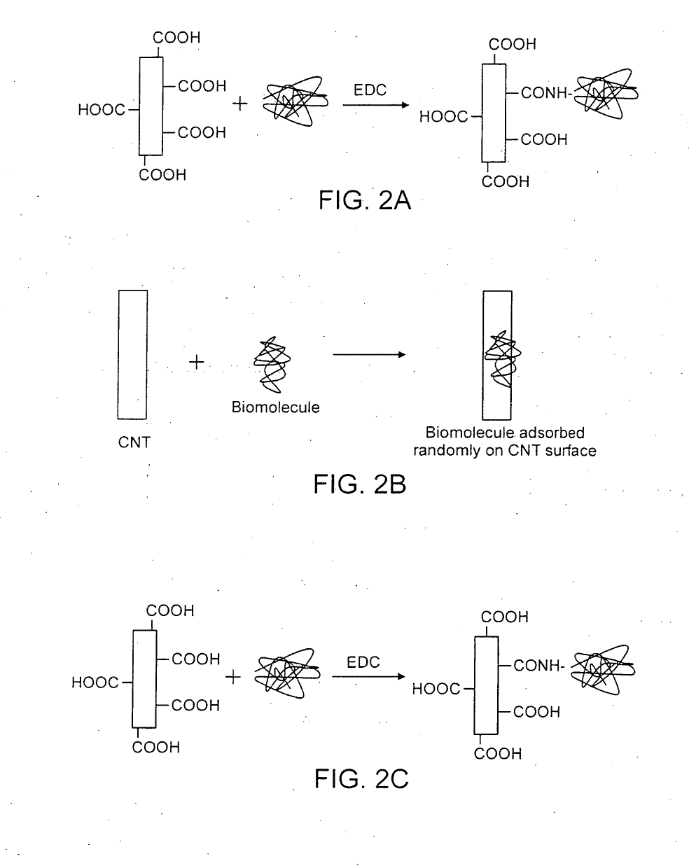

FIG. 3 illustrates the process of preparing carbon nanotubes according the method described below. Purified HIPCO single-walled carbon nanotubes (SWNTs) were purchased from Carbon Nanotechnologies Inc. and acid-treated using a 3:1 mixture of sulfuric acid (50.4 g of 98% w/v concentrated sulfuric acid diluted to 100 ml with distilled water) and nitric acid (10.5 g of 70% w/v concentrated nitric acid diluted to 33 ml with distilled water) as follows: first, 5.3 mg of purified HIPCO SWNTs were taken with 133 ml of the acid mixture in a 250 ml round bottom flask and sonicated (Branson Sonicator) for 20 minutes; then, the contents of the flask were refluxed for 90 minutes at a variator setting of 70-80 with stirring at setting 6-7 (using a magnetic stirrer); following this step, the mixture was cooled for another 45 minutes with stirring, diluted with 250 ml distilled water and filtered using a 0.45 pm Teflon membrane. The resulting SWNTs are high in purity and chemically functionalized with carboxylic acid groups (COOH), which aids to covalently bond desired molecules (such as DNA, proteins, drugs, antibodies) to the nanotubes for various applications.

The nanotubes entrapped on the membrane were further subjected to four washes of distilled water, one wash of ammonium hydroxide solution (to solubilize amorphous carbon residues entrapped in the filter membrane), and another four washes of distilled water. Then, the nanotubes were resuspended in 20 ml distilled water by sonicating the filter membrane in the water for 10 minutes followed by discarding the membrane. This constituted the nanotube stock solution.

The nanotube stock solution was diluted in the ratio of 2.14 ml distilled water per 1 ml nanotube stock solution and sonicated for 5 minutes prior to adding to capsule surfaces or cores. Large capsules (2 mm diameter) were prepared by forcing a 1.65% w/v sodium alginate solution through a syringe fitted with a 21 gauge needle, manually, and coagulating the resulting drops in a 0.1M calcium chloride solution for at least 10 minutes, to obtain calcium alginate (A) capsules. For alginate capsules with nanotubes embedded inside, 2 ml 2.475% w/v sodium alginate solution was diluted to 1.65% w/v using 1 ml of diluted nanotube solution and formed into the capsules as before. Subsequent alternating layers of polylysine (P) and alginate (A) were incorporated onto the alginate capsules by incubating the capsules in 0.1% w/v poly-1-lysine and 0.1% w/v sodium alginate solutions respectively for 10 minutes each on a rotary shaker at about 120 rpm at room temperature.

Nanotube layers were added to either the plain alginate capsule surfaces or in between the outer polylysine and alginate membranes by incubating the capsules in the diluted nanotube solution for 10 minutes on a rotary shaker at 120 rpm. The addition of each A, P or nanotube layer to the capsules was followed by three washes (1 ml per wash) of physiological solution (0.9% w/v aqueous NaCl solution) to simulate the conditions in which protein solutions were incorporated into alginate capsules. The physiological solution washes also helped remove, as far as possible, any excess unbound calcium ions present in the capsules.

The prepared capsules were finally stored in 0.1M CaCl₂ solution to preserve their integrity until characterization.

The surface morphology of the capsules was characterized using optical microscopy (LOMO microscope, Russia), FE-SEM (after critical point drying or CPD of the capsules) and Raman spectroscopy. The CPD-dried capsules were coated with gold-palladium for about 45 seconds using a Hummer VI instrument, prior to FE-SEM. The presence of carbon nanotubes in the capsule cores or outer membranes was spectroscopically confirmed using Raman spectroscopy.

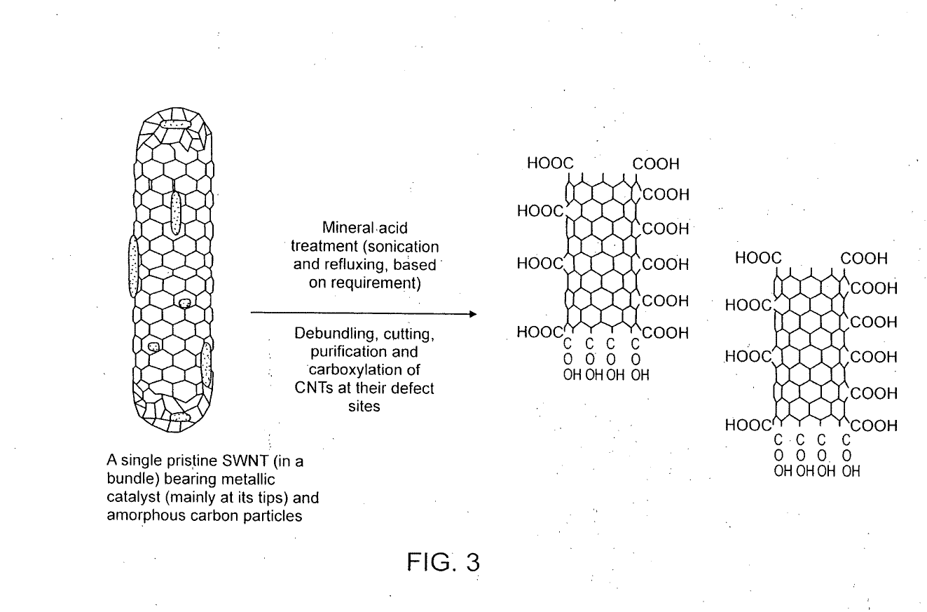

The results presented in FIGS. 4(a) through 4(e) below depict optical photomicrographs of the surfaces of alginate and alginate-polylysine capsules incorporating carboxylated (acid-treated) purified HIPCO single-walled carbon nanotubes (from Carbon Nanotechnologies Inc., USA) on their surfaces or inside their cores. The nanotubes were visible, where present on the capsule surfaces, as black patches. FIG. 4(a) shows alginate, FIG. 4(b) shows alginate with nanotubes on capsule surface, FIG. 4(c) shows alginate-polylysine-alginate control, FIG. 4(d) shows alginate-polylysine-alginate with nanotubes between the outermost two layers, FIG. 4(e) shows alginate with nanotubes embedded in its core.

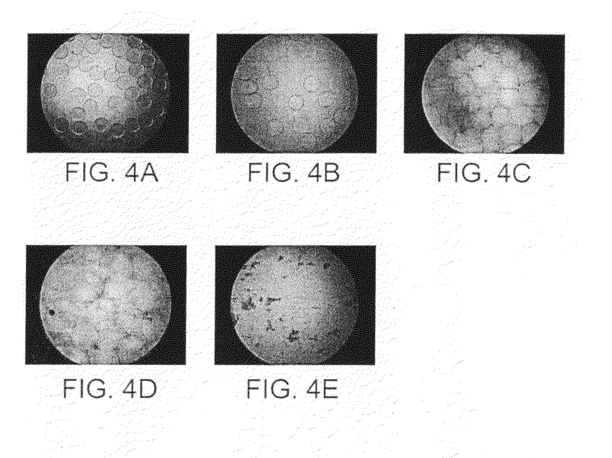

The results presented in FIGS. 5(a) through 5(f) below depict scanning electron micrographs (SEM) of the surfaces of alginate and alginate-polylysine capsules incorporating carboxylated (acid-treated) single-walled carbon nanotubes on their surfaces or inside their cores. An SEM image of a dried mesh of nanotubes (FIG. 5(f)) is also shown for comparison and to provide an idea of how the nanotubes themselves look under high magnification. FIG. 5(a) shows alginate, FIG. 5(b) shows alginate with nanotubes on capsule surface, FIG. 5(c) shows alginate-polylysine-alginate, FIG. 5(d) shows alginate-polylysine-alginate with nanotubes between the outermost two layers, FIG. 5(e) shows alginate with nanotubes embedded in its core, and FIG. 5(f) shows aried mesh of nanotubes on a metal surface.

In FIGS. 5(a) and 5(c) needle-like structures were visible even though nanotubes were not incorporated onto their surfaces. This is most probably due to the in-situ formation of crystals of calcium and sodium salts during the critical point drying (CPD) process preceding the SEM studies. The fibrous structures present in FIG. 5(c) could have been cross-linked alginate-polylysine polymeric fibers, while those in FIG. 5(d) could have been a combination of both the polymeric fibers and the nanotubes. In the case of FIG. 5(e), the nanotubes seemed to be protruding from inside the capsule core and through the capsule surface. This could have been due to the shrinkage of the capsule during the CPD process prior to SEM.

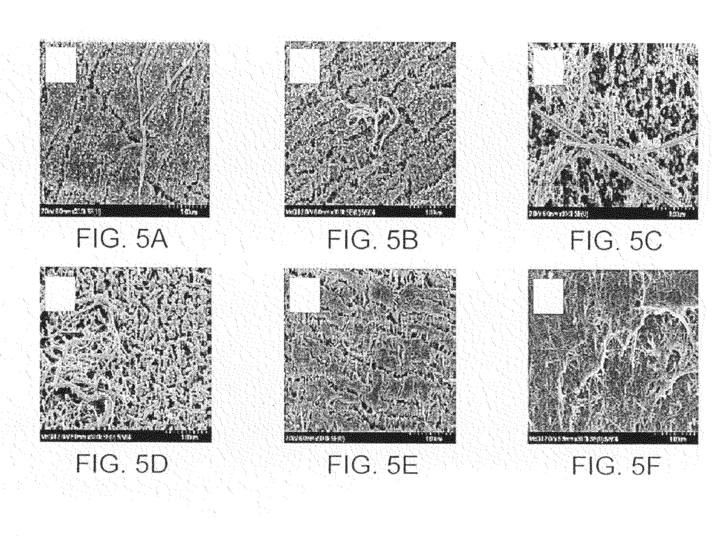

The presence of carbon nanotubes in the capsule cores or outer membranes (in the wet condition) was confirmed by Raman spectroscopy. A Renishaw Raman microscope with a 514.5 nm line from an argon ion laser operating at 24 mW power, and a Zeiss microscope with a 50× objective was used. Raman spectra were taken from at least three different positions of the samples to verify the reproducibility and consistency of the data. Three accumulations for each position, with an accumulation time of 10 seconds were maintained for all the Raman measurements. Prominent nanotube signals (D band, G band and the second order G band) at −1330, −1585, and −2660 cm⁻ respectively were seen in the case of the samples CNT, ACNT and ACNT Embed. A faint G band was observed at 1587.11 cm⁻ in the case of the sample APCNTA, indicating the presence of nanotubes in it. The Raman signals for nanotubes embedded in APCNTA were not prominent. This is due to the outer alginate and polylysine layers of the sample restricting the Raman intensities. As expected, no nanotubes peaks were observed in the case of the control samples A and APA. In the case of the samples containing nanotubes on the surface, the Raman spectra were acquired by focusing the laser beam of the instrument directly onto the dark patches (the nanotubes) on the capsule surfaces. For the other samples, the Raman study was performed on randomly chosen sites. FIG. 6 depicts sample Raman spectra of some of the capsule samples, correlating to the ones shown in FIGS. 4 and 5.

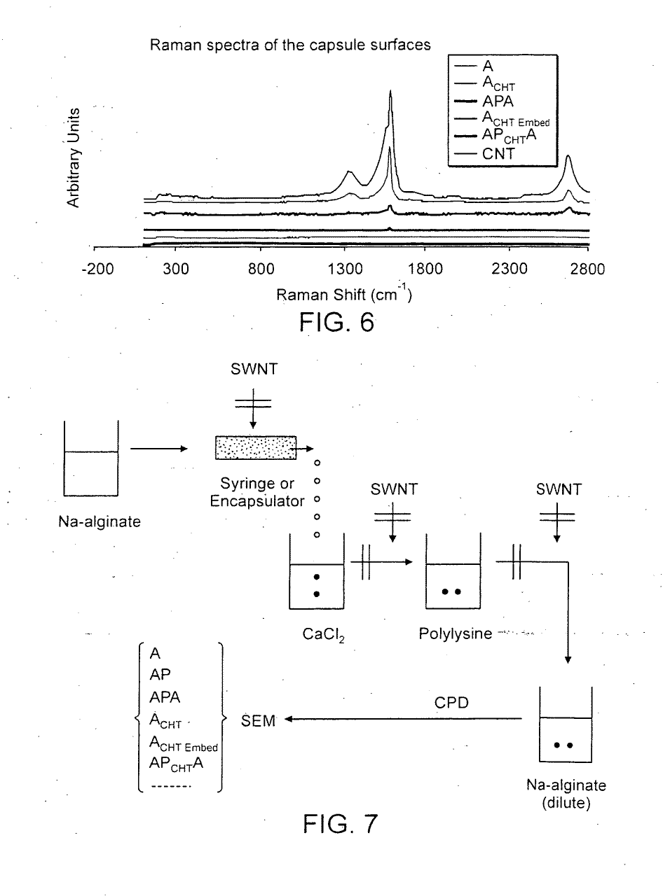

FIG. 7 depicts the process used for making the capsules used for the above characterization studies. The twin lines intersecting the arrows indicate that either the nanotubes were incorporated onto the capsule surfaces or into the capsule cores, or they were not, depending on the type of sample desired. In FIG. 7, the capsules were washed 3 times (1 ml per wash) with 0.09% w/v NaCl solution (termed “physiological solution”) after each treatment (with SWNT or polylysine or Na-alginate).

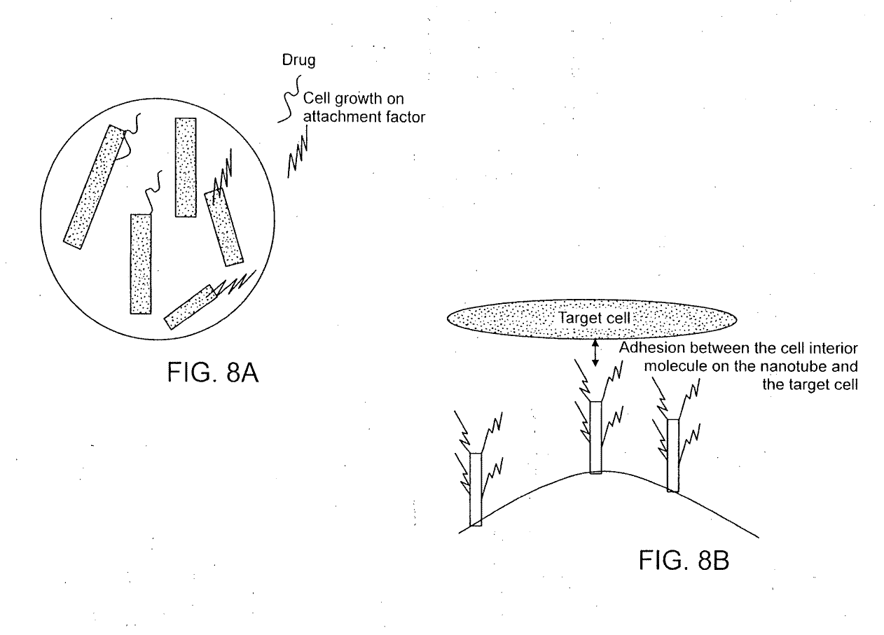

The nanotubes can be suitably functionalized with a biochemical entity, prior to incorporation into the capsule surfaces or cores. The nanotubes to be placed on the capsule surface can be functionalized with suitable cell adhesion molecules to promote attachment between the capsule device and the target cells, to achieve targeted delivery of drugs or cells as demonstrated in Example 2. The nanotubes to be placed inside the capsule core can be functionalized with the drug chosen for delivery, or with biochemicals essential for maintaining the viability of the encapsulated cells, such as cell growth or attachment factors. FIG. 8(a) illustrates functionalized nanotubes placed inside a capsule, while FIG. 8(b) illustrates nanotubes embedded on the surface of a capsule of gel slab.

Alternately, the surface or core nanotubes can be used to adsorb body toxins in the same manner as activated carbon. The above process can be stopped before the CPD step for biochemical characterization studies or drug/cell delivery applications or even optical microscopy or Raman spectroscopy.

This process can be extended to incorporate other gel-forming materials, and multiwalled carbon nanotubes, silica or peptide nanotubes or other types of nanostructured materials. Instead of forming capsules as demonstrated by the syringe/encapsulator shown in the figure, the alginate gels can be formed into several well-defined geometries and films, depending on the desired application or characterization method. By extending the above process, additional outer layers of polylysine, nanotubes and alginates can also be added to the capsules or gels of defined geometries.

### Example 11

### Delivery of APC Gene to Colon Cancer Cells Using Functionalized SWNTs

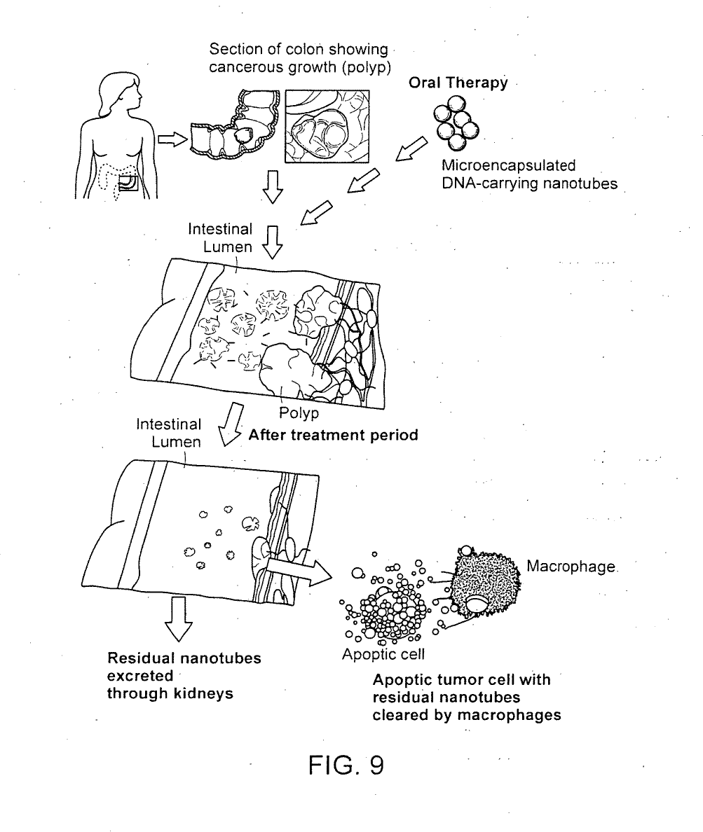

Carbon nanotubes have been shown to have the potential to carry molecules and enter mammalian and bacterial cells and are not toxic by themselves. (See Gao, L. et al. Carbon nanotube delivery of the GFP gene into mammalian cells. Chembiochem. 7, 239-242 (2006); Pantarotto, D. et al. Functionalized carbon nanotubes for plasmid DNA gene delivery. Angewandte Chemie-International Edition 43, 5242-5246 (2004); Singh, R. et al. Binding and condensation of plasmid DNA onto functionalized carbon nanotubes: toward the construction of nanotube-based gene delivery vectors. J. Am. Chem. Soc. 127, 4388-4396 (2005); Kam, N. W. S., Jessop, T. C., Wender, P. A. & Dai, H. J. Nanotube molecular transporters: Internalization of carbon nanotube-protein conjugates into mammalian cells. Journal of the American Chemical Society 126, 6850-6851 (2004); Pantarotto, D., Briand, J. P., Prato, M. & Bianco, A. Translocation of bioactive peptides across cell membranes by carbon nanotubes. Chemical Communications 16-17 (2004).) The proposed microcapsule carbon nanotube device can be functionalized with various DNA molecules for successful use in gene therapy applications and used as an oral formulation in cancer therapy. FIG. 9 represents the concept of using the microcapsule carbon nanotube device as an oral formulation to treat colorectal cancer by selectively delivering the APC gene to the cancer cells.

The feasibility of transfection of cells using the proposed device was tested using SW480 colon cancer cells and pAcGFP1-C1 plasmid, a plasmid carrying the green fluorescent protein gene. The plasmid on expression inside the cells fluoresces with a bright green color. The SW480 cells were cultivated in Leibovitz's media at 37° C. and exposed to the carbon nanotube device functionalized with the pAcGFP-C1 plasmid. Lipofectamine 2000 (a commonly used cationic liposome transfection reagent) was used as control for transfection. The cells were observed 24 hours after transfection under a confocal fluorescent microscope to visualize the GFP expression. The images were recorded at 40× magnification and an excitation wavelength of 488 nm.

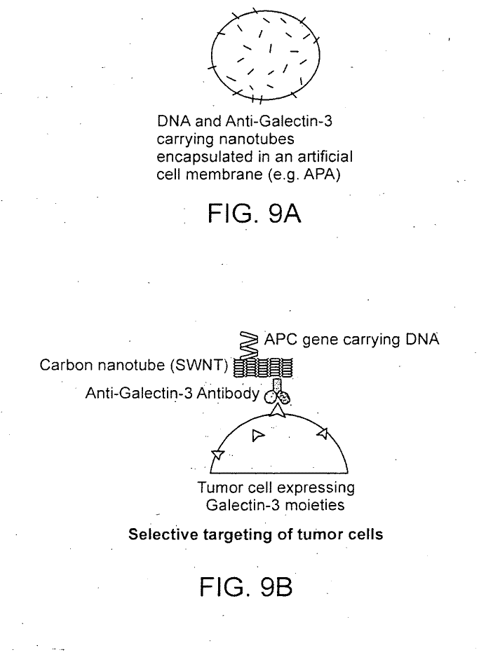

The confocal microscopy images (FIG. 10) indicated that the carbon nanotube gene delivery system was able to transfect the SW480 cells with the gene. The presence of cells expressing the protein in all treatments showed the efficacy of the gene carrier systems in transporting the plasmid across the cell membrane.

### Example 12

### Use of the Devices of the Present Invention for Drug Delivery

### Targeted Delivery of Drugs to Treat Breast Cancer

The microcapsule carbon nanotube device can be used to deliver therapeutic drugs to treat cancer. One of the major limitations of conventional chemotherapy for cancer patients is the toxicity associated with the lack of specificity of drugs for tumor cells. It was reported that SWNTs can be functionalized with folate moieties, thus making it a convenient targeted delivery device. The proposed microcapsule carbon nanotube device can be functionalized with drug-folate conjugates and targeted to membranous folate receptors and serve as a site-specific drug-delivery system for breast cancer. (See Fujimori, M. Genetically engineered bifidobacterium as a drug delivery system for systemic therapy of metastatic breast cancer patients. Breast Cancer 13, 27-31 (2006); Kam, N. W., O'Connell, M., Wisdom, J. A. & Dai, H. Carbon nanotubes as multifunctional biological transporters and near-infrared agents for selective cancer cell destruction. Proc. Natl. Acad. Sci. U.S.A 102, 11600-11605 (2005).)

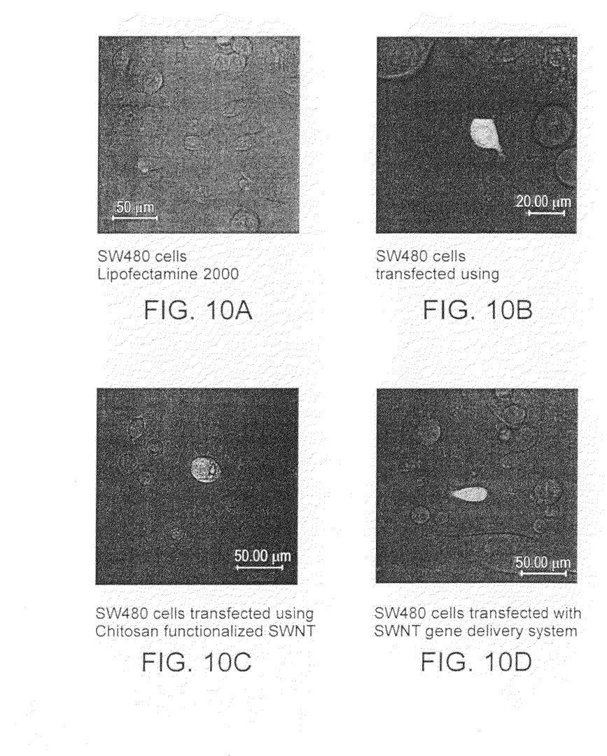

FIG. 11 illustrates using proposed novel microcapsule carbon nanotube device for targeted delivery of drugs to treat breast cancer. The device is functionalized with anti-cancer drugs like taxol or nascopine and folate molecules to selectively target cancer cells.

Use of Formulation for Targeted Delivery of Drugs for Cancer Therapy Functionalization of Carbon Nanotubes with Colon Cancer Specific Drugs

Targeted delivery of cancer drugs is an essential strategy in treating cancer. To investigate the formulation for cancer therapy applications, functionalization of carbon nanotube device with albumin-bound cancer drug (e.g paclitaxel) was performed to be delivered using microcapsules at the target site. This was achieved by the diimide-activated amidation reaction with serum albumin as described earlier. (See Huang, W. J. et al. Attaching proteins to carbon nanotubes via diimide-activated amidation. Nano Letters 2, 311-314 (2002).) Paclitaxel dissolved in anhydrous ethanol was added to the aqueous solution of human serum albumin (HSA). After 90 min of equilibration at 22° C. precipitated paclitaxel was removed by centrifugation (13 000 g, 15 min). SWNTs and 1-ethyl-3-(3-dimethylaminopropyl)carbodiimide (EDAC) are added to an aqueous KH₂PO₄ buffer solution. After sonicating the mixture for 2 hours, HSA with bound paclitaxel is added. The resulting mixture was stirred at room temperature for 12 hours and then the unbound albumin-drug conjugate was removed by filtration using a centrifugal filter with MWCO 30 kD. The Bio-Rad DC protein assay was conducted before and after centrifugation to determine the extent of binding of the albumin-drug conjugates to the carbon nanotubes. The samples were analyzed using a Cary 100 Bio UV-vis spectrophotometer at 650 nm.

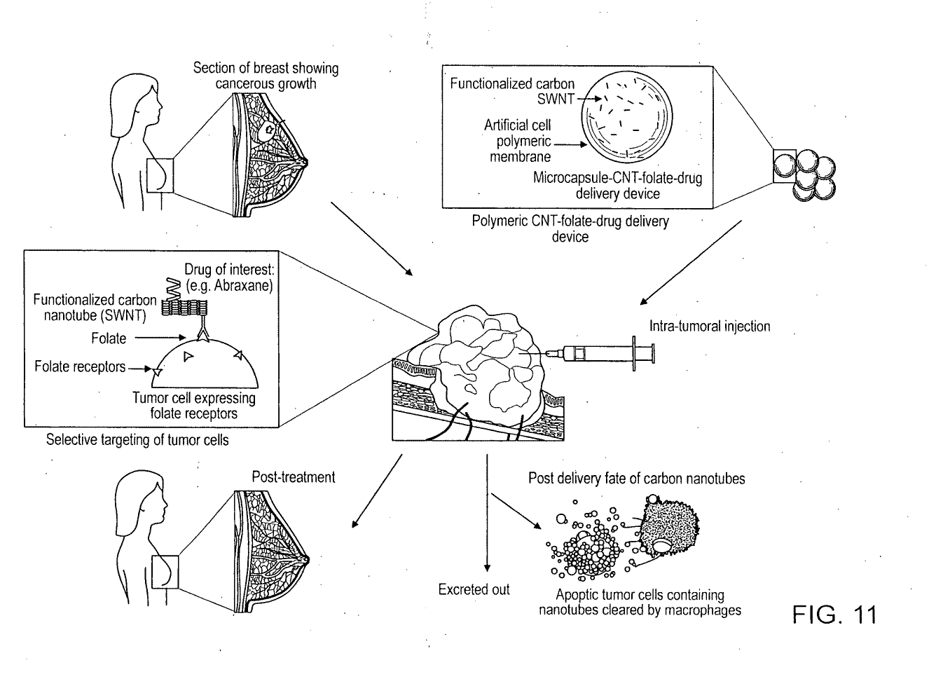

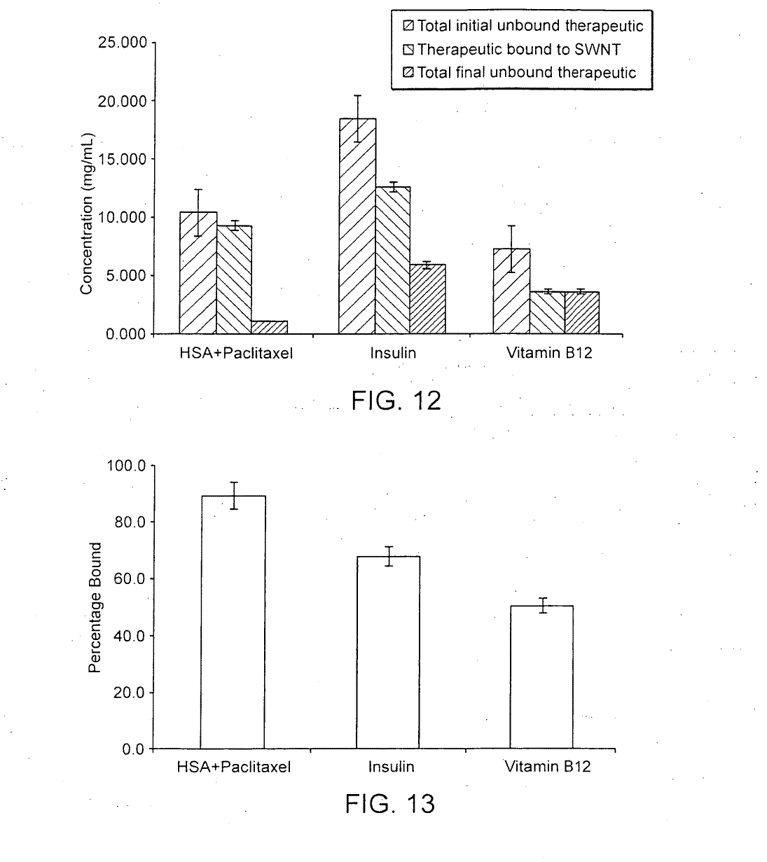

Results show (FIG. 12 and FIG. 13) that the albumin bound drug was effectively bound to the carbon nanotube device. A binding efficiency approximately 90% was achieved. Therefore, an important aspect of this invention that cancer drugs can be delivered at targeted sites using the novel microcapsule carbon nanotube formulation.

### Use of Formulation for Targeted Delivery of Insulin for Use in Diabetes and Other Clinical Applications

Insulin is the most common drug therapy for patients with insulin-dependent diabetes mellitus. At present, the normal mode of delivery of insulin to patients is via intramuscular, subcutaneous or intravenous injections. Oral delivery is far more popular with patients especially for long-term treatment, however Insulin being a peptide drug is still a challenge for oral dosage, due to enzymatic and/or metabolic degradation in the gastrointestinal tract. The proposed microcapsule carbon nanotube device can be used to overcome these shortcomings and effectively deliver Insulin.

The functionalization of SWNTs with insulin was based on the diimide-activated amidation of nanotube-bound carboxylic acids. For this functionalization, carbon nanotubes, EDAC were mixed at a certain proportion. After the mixture was sonicated for 2 hours, insulin was added. The resulting mixture was stirred in an ice bath for 12 hours to obtain SWNT-insulin conjugates. Unbound insulin was removed by filtration using a centrifugal filter with MWCO 30 kD. Extent of protein binding was determined using the Bi-Rad DC protein assay and UV-vis spectrophotometry. The results obtained show (FIG. 12) that the formulation can be successfully used to functionalize insulin molecules on carbon nanotubes and use for targeted delivery such as oral delivery.

### Targeted Delivery of Vitamins

In many diseases such as wound healing, local delivery of vitamins is required. We have investigated this capacity of the present formulation for the same. For this, single walled carbon nanotubes were functionalized with Vitamin B12 based on the diimide-activated amidation of nanotube-bound carboxylic acids. Single walled carbon nanotubes were mixed with EDAC and sonicated for 2 hours. An aqueous solution of Vitamin B12 was added to this and the resulting mixture was stirred in an ice bath for 12 hours to obtain Vitamin B12 functionalized SWNTs. The mixture was filtered using a centrifugal filter to separate unbound Vitamin B12 from the nanotube solution. The extent of binding of Vitamin B12 on nanotubes was determined using the Cary 100 Bio UV-vis spectrophotometer at 361 nm. Results show (FIG. 12 and FIG. 13) that there was a 50% binding efficiency of vitamin B12 to carbon nanotubes, hence allowing the application of the formulation successfully in targeted delivery of vitamins.

### Example 13

### Preparation of Gel Matrix/Slab Containing Microcapsule Carbon Nanotube Device or Gel Slab Containing Functionalized Carbon Nanotubes

Slabs of gel can be produced using plastic cylinders or boxes of various dimensions (depending on the application) covered on both faces by dialysis membranes with a diffusion threshold of 12,000-14,000 Da. However, these gel slabs were prepared for immobilizing bacterial cells and were made with alginate only or alginate-hyaluronate complexes.

Our novel polymeric gel matrix/slab would contain microcapsule incorporated or free carbon nanotubes functionalized with therapeutics for various applications. An aqueous solution of a hyrdogel forming material (e.g. sodium alginate) and free or encapsulated CNTs functionalized with drug, DNA, proteins or other therapeutic molecules, is poured into the required geometry and dialysed against a calcium chloride solution (0.05M) for gel formation. Simultaneous removal of air with a syringe while pouring the solution is done to get rid of air bubbles.

### Example 14

### Functionalization of Carbon Nanotube for Colon Cancer Targeted DNA Delivery

Two types of CNTs (SWNT and MWNTs) are used. The experiments CNTs are made hydrophilic by ultrasonication in a sulfuric and nitric acid mixture (3:1) for a suitable period of time to functionalize CNTs sidewalls and tips with hydrophilic carboxylic acid and hydroxyl groups to form stable suspensions in water and to remove unwanted iron catalyst. Once this is achieved, nanotubes are used for subsequent functionalization with DNA. Nanotube-DNA functionalization is achieved in a way analogous to previously published methods. Non-covalent route is selected for this, for the sake of simplicity and to ensure the functional integrity of the plasmid DNA. Cancer suppressing normal (wild type) APC gene, which has potential to prevent or delay the onset of colon cancer, is used. For designing colon cancer cell targeted nanotube, APC DNA nanotubes are further functionalized with fluorescent tag galectin-3 monoclonal antibody using the procedure described by others for CNTs functionalizations with monoclonal antibodies. This allows colon cancer cell specific targeted delivery. Expression of Galectin-3, a beta-galactoside-binding protein, has been linked with progression of colon cancer and has been projected as possible target for delivery of therapeutics to cancerous cells. In addition, anti-galectin-3 monoclonal antibody with a fluorescent tag has been used to target SW 480 colon cancer cells in vitro. The CNT-wild type Apc plasmid-(Anti-Galectin-3) conjugates are effectively targetting galectin-3 expressed at the cancer cells and serve as exibits colon cancer therapy.

### Example 15

### Method for Functionalization of Carbon Nanotubes with Folate and Albumin-Paclitaxel Conjugates for Breast Cancer Targeted Drug Delivery

Single walled carbon nanotubes (SWNT) are functionalized with a folate moiety. They are then further functionalized with the albumin-paclitaxel conjugate by the diimide-activated amidation reaction with serum albumin. SWNTs and 1-ethyl-3-(3-dimethylaminopropyl)carbodiimide (EDAC) is added to an aqueous KH₂PO₄ buffer solution.

After sonicating the mixture for 2 hours, HSA with bound paclitaxel is added. The resulting mixture is stirred at room temperature for 24 h and then is dialyzed (membrane mol. weight cutoff of 12000 kD). Following the dialysis for 3 days, the mixture is centrifuged at 7800 rpm to separate the insoluble nanotubes. Free HSA-paclitaxel conjugate in the solution is removed via further dialysis in a membrane tubing of a larger pore size (cutoff molecular weight 100,000 kD) for 3 days. The SWNT-folate-HSA-paclitaxel conjugate sample is obtained by evaporation. This device enables the specific and targeted delivery of paclitaxel molecules to tumors on breast cancer sites—a new strategy for breast cancer drug delivery. Ligand-mediated targeting of anticancer drugs to target receptors expressed selectively or over expressed on tumor cells can be an effective strategy for improving the therapeutic indices of breast anticancer drugs.

### EQUIVALENTS

While this invention has been particularly shown and described with references to preferred embodiments thereof, it will be understood by those skilled in the art that various changes in form and details may be made therein without departing from the scope of the invention encompassed by the appended claims.

What is claimed is:

1. A carbon nanotube (CNT) device, comprising:
a gel matrix that includes microcapsules; and
carbon nanotubes incorporated into said gel matrix.

2. The CNT device of claim 1, wherein the CNTs are incorporated on the surface of said microcapsules.

3. The CNT device of claim 1, wherein the CNTs are incorporated inside said microcapsules.

4. The CNT device of claim 1, wherein the CNTs are singlewalled carbon nanotubes (SWNTs).

5. The CNT device of claim 1, wherein the CNTs are multiwalled carbon nanotubes (MWNTs).

6. The CNT device of claim 1, wherein the CNTs are branched SWNTs

7. The CNT device of claim 1, wherein the CNTs are branched MWNTs.

8. The CNT device of claim 1, wherein the CNTs are functionalized by attaching a coupling agent.

9. The CNT device of claim 1, wherein the coupling agent is selected from the group consisting of folate, albumin, and 1-ethyl-3-(3-dimethylaminopropyl) carbodiimide (EDAC).

10. The CNT device of claim 1, further including a biologically active ingredient selected from a small molecule, a peptide molecule and a DNA molecule.

11. The CNT device of claim 10, wherein the CNT is functionalized and said functionalized CNT is attached to the biologically active ingredient.

12. The CNT device of claim 10, wherein the biologically active ingredient is included within the CNT.

13. The CNT device of claim 10, wherein the CNT is functionalized and said functionalized CNT is attached to a DNA molecule.

14. The CNT device of claim 10, wherein the CNT is functionalized and said functionalized CNT is attached to an antibody molecule.

15. The CNT device of claim 1, wherein the gel matrix comprises a material selected from the group consisting of alginate-poly-L-lysine-alginate (APA), cellulose acetate phthalate (CAP) coated with beeswax, calcium alginate and κ-carrageenan-locust bean gum gel, gellan-xanthan, agarose, alginate chitosan poly-L-lysine, poly(lactide-co-glycolides), carrageenan, poly(D,L-lactic acid), alginate-poly-L-lysine, starch polyanhydrides, liposomes, polymethacrylates, polyamino acids, and enteric coating polymers.

16. The CNT device of claim 1, wherein the gel matrix comprises alginate-poly-L-lysine-alginate (APA).

17. A pharmaceutical composition, comprising one or more carbon nanotube (CNT) device in a pharmaceutically acceptable carrier, said device including
a gel matrix that includes microcapsules; and
carbon nanotubes incorporated into said gel matrix.

18. The pharmaceutical composition of claim 17, wherein the CNTs are functionalized by attaching a coupling agent.

19. The pharmaceutical composition of claim 17, wherein the coupling agent is selected from the group consisting of folate, albumin, and 1-ethyl-3-(3-dimethylaminopropyl)carbodiimide (EDAC).

20. The pharmaceutical composition of claim 17, further including a biologically active ingredient selected from a small molecule, a peptide molecule or a DNA molecule.

21. The pharmaceutical composition of claim 20, wherein the CNT is functionalized and said functionalized CNT is attached to the biologically active ingredient.

22. The pharmaceutical composition of claim 20, wherein the biologically active ingredient is included within the CNT.

23. The pharmaceutical composition of claim 20, wherein the functionalized CNTs are attached to a DNA molecule.

24. The pharmaceutical composition of claim 20, wherein the functionalized CNTs are attached to an antibody molecule.

25. The pharmaceutical composition of claim 17, wherein the CNTs are incorporated on the surface of said microcapsules.

26. The pharmaceutical composition of claim 17, wherein the CNTs are incorporated inside said microcapsules.

27. The pharmaceutical composition of claim 17, wherein the gel matrix comprises a material selected from the group consisting of alginate-poly-L-lysine-alginate (APA), cellulose acetate phthalate (CAP) coated with beeswax, calcium alginate and κ-carrageenan-locust bean gum gel, gellan-xanthan, agarose, alginate chitosan poly-L-lysine, poly(lactide-co-glycolides), carrageenan, poly(D,L-lactic acid), alginate-poly-L-lysine, starch polyanhydrides, liposomes, polymethacrylates, polyamino acids, and enteric coating polymers.

28. The pharmaceutical composition of claim 17, wherein the gel matrix comprises alginate-poly-L-lysine-alginate (APA).

29. A method of treating a patient suffering from toxins, comprising administering to said patient an effective amount of a composition comprising carbon nanotube (CNT) devices or pharmaceutically acceptable salts, or solvates thereof, said device including:
a gel matrix that includes microcapsules; and
carbon nanotubes incorporated into said gel matrix.

30. A method of selective removal of enzymes in a patient in need therefor, said method comprising administering to said patient an effective amount of a composition comprising carbon nanotube (CNT) devices or pharmaceutically acceptable salts, or solvates thereof, said device including:
a gel matrix that includes microcapsules; and
carbon nanotubes incorporated into said gel matrix.

31. The method of claim 30, wherein the composition is administered orally.

32. The method of claim 30, wherein the composition is administered parenterally.

33. The method of claim 30, wherein the CNTs are functionalized by attaching a coupling agent moiety.

34. The method of claim 30, wherein the coupling agent is selected from the group Consisting of folate, albumin, and 1-ethyl-3-(3-dimethylaminopropyl) carbodiimide (EDAC).

35. The method of claim 30, further including a biologically active ingredient selected from a small molecule, a peptide molecule or a DNA molecule.

36. The method of claim 35, wherein the CNT is functionalized and said functionalized CNT is attached to the biologically active ingredient.

37. The method of claim 35, wherein the biologically active ingredient is included within the CNT.

38. The method of claim 35, wherein the functionalized CNTs are attached to a DNA molecule.

39. The method of Claim 35, wherein the functionalized CNTs are attached to an antibody molecule.

40. A composition of matter, comprising:
a gel slab that includes microcapsules; and
functionalized carbon nanotubes incorporated into said gel slab,
wherein the functionalized carbon nanotubes are selected from single wall or multiple carbon nanotubes.

41. A composition of matter, comprising:
a gel matrix that includes microcapsules; and
particles incorporated into said gel matrix,
wherein the particles include carbon nanotubes chemically altered by oxidation.

42. A method for treating cancer in a patient, comprising administering to the patient an effective amount of a composition comprising carbon nanotube (CNT) devices including:
a gel matrix that includes microcapsules; and
carbon nanotubes or pharmaceutically acceptable therapeutic materials thereof, incorporated into said gel matrix and carbon nanotubes.

43. The method of claim 42, wherein the cancer is colon cancer.
